SEQUENCE LISTING (23 sequences)

SEQ ID NO: 1 - protein, 399 aa, Pseudomonas putida
  SITE (1)..(399): alkB-type oxidoreductase
EKHRVLDSAPEYVDKKKYLWILSTLWPATPMIGIWLANETGWGIFYGLVLLVWYGALPLLDAMFGEDFNNPPEEVVPKLEKERYYRVLTYLTVPMHYAALIVSAWWVGTQPMSWLEIGALALSLGIVNGLALNTGHELGHKKETFDRWMAKIVLAVVGYGHFFIEHNKGHHRDVATPMDPATSRMGESIYKFSIREIPGAFIRAWGLEEQRLSRRGQSVWSFDNEILQPMIITVILYAVLLALFGPKMLVFLPIQMAFGWWQLTSANYIEHYGLLRQKMEDGRYEHQKPHHSWNSNHIVSNLVLFHLQRHSDHHAHPTRSYQSLRDFPGLPALPTGYPGAFLMAMIPQWFRSVMDPKVVDWAGGDLNKIQIDDSMRETYLKKFGTSSAGHSSSTSAVAS

SEQ ID NO: 2 - protein, 295 aa, Pseudomonas sp.
  SITE (1)..(295): enzyme E1a
MRRESLLVSVCKGLRVHVERVGQDPGRSTVMLVNGAMATTASFARTCKCLAEHFNVVLFDLPFAGQSRQHNPQRGLITKDDEVEILLALIERFEVNHLVSASWGGISTLLALSRNPRGIRSSVVMAFAPGLNQAMLDYVGRAQALIELDDKSAIGHLLNETVGKYLPPRLKASNHQHMASLATGEYEQARFHIDQVLALNDRGYLACLERIQSHVHFINGSWDEYTTAEDARQFRDYLPHCSFSRVEGTGHFLDLESKLAAVRVHRALLEHLLKQPEPQRAERAAGFHEMAIGYA

SEQ ID NO: 3 - protein, 346 aa, Burkholderia sp.
  SITE (1)..(346): enzyme E1b
MRGSGEWVAAAARVRQGGQIAREGGYVEASIKGAGSAHLPSRCGRYAMPIEKQVVALPSGLKVHVERHVFDPAFETVILVNGALATTASFGQTIRYLGERVNAVCFDLPYAGQSRQHNPGEYILTKDDEVEILLHLAERFEPSFLLSVSWGGVASLFALARGCASVRRAVIASFSPFLNDAMTDYVTRARDHIAAGENLKAAQLLNDTVGRYLPRIMKLYNYRYLTKLPRTEQDQVAFHVDQILSMRPEQYLPEFRQIGCAVKFINGELDEYTTASDVRRLAAYVRRAEFATIRQAGHFLDLEGRQQQEQLRAAILGFFGDERASAARDDAQDETLAPLGQLPALS

SEQ ID NO: 4 - protein, 295 aa, Pseudomonas sp.; identical to SEQ ID NO: 2
  SITE (1)..(295): enzyme E1c

SEQ ID NO: 5 - protein, 295 aa, Pseudomonas sp.
  SITE (1)..(295): enzyme E1d
MRRESLLVSVCKGLRVHVERVGQDPGRSTVMLVNGAMATTASFARTCKCLAEHFNVVLFDLPFAGQSRQHNPQRGLITKDDEVEILLALIERFEVNHLVSASWGGISTLLALSRNPRGIRSSVVMAFAPGLNQAMLDYVGRAQALIELDDKSAIGHLLNETVGKYLPQRLKASNHQHMASLATGEYEQARFHIDQVLALNDRGYLACLERIQSHVHFINGSWDEYTTAEDARQFRDYLPHCSFSRVEGTGHFLDLESKLAAVRVHRALLEHLLKQPEPQRAERAAGFHEMAIGYA

SEQ ID NO: 6 - protein, 295 aa, Pseudomonas sp.
  SITE (1)..(295): enzyme E1e
MRRESLLVTVCKGLRVHVERVGQDPGRDTVMLVNGAMATTASFARTCKCLAEHFNVVLFDLPFAGQSRQHNPQRGLITKDDEVEILLALIERFAVNHLVSASWGGISTLLALSRNPRGVRSSVVMAFAPGLNQAMLDYVGRAQELIELDDKSAIGHLLNETVGKYLPPRLKASNHQHMASLATGEYEQARFHIDQVLALNDRGYLSCLGQIQSHVHFINGSWDEYTTAEDARQFRDYLPHCSFSRVEGTGHFLDLESKLAAARVHRALLEHLLAQPEPWRSEQAAGFHEMAIGYA

SEQ ID NO: 7 - protein, 426 aa, Pseudomonas sp.
  SITE (1)..(426): Enzyme E2a
MHAILIAIGSAGDVFPFIGLARTLKLRGHRVSLCTIPVFRDAVEQHGIAFVPLSDELTYRRTMGDPRLWDPKTSFGVLWQTIAGMIEPVYEYVSAQRHDDIVVVGSLWALGARIAHEKYGIPYLSAQVSPSTLLSAHLPPVHPKFNVPEQMPLAMRKLLWRCIERFKLDRTCAPDINAVRRKVGLETPVKRIFTQWMHSPQGVVCLFPAWFAPPQQDWPQPLHMTGFPLFDGSIPGTPLDDELQRFLDQGSRPLVFTQGSTEHLQGDFYAMALRALERLGARGIFLTGAGQEPLRGLPNHVLQRAYAPLGALLPSCAGLVHPGGIGAMSLALAAGVPQVLLPCAHDQFDNAERLVRLGCGMRLGVPLREQELRGALWRLLEDPAMAAACRRFMELSQPHSIACGKAAQVVERCHREGDARWLKAAS

SEQ ID NO: 8 - protein, 493 aa, Burkholderia sp.
  SITE (1)..(493): Enzyme E2b
MDAGRIGLHDAAAAGRIGMTEAFASRARCSAAALAAGGRAPAGDGRSGSNRAAANGSVDCRAGWNDEAMAKVIVTAIGSAGDVHPLLGVSRALSARGHEVVFCTHAPFEAAVRASGFAFVPVGTAEDYVRAMADPALWDPRTSFKTLWRVIAPVVRPHFEVLRALSDADTVLVGTLWAFSARLMQERFGTRYVSVQVSPSTLLSAHAPPTHKRLTIPKGLPLAVKAGLMTLIERQVLDRVCGPELNAARQALGLAPAKRILGRWLHSTDGVLCLFPSWFAPAQPDWPANHLQSGFPLFNDAGPAQADAELEAFVASGEAPVVFTAGSTLVDGRTYEHAVTQVLQATGVRGILLAPDAPDAPAASDGAALLKRRYVPLAALLPRCRALVHHGGIGTASLAYAAGVPQVVTPFAHDQFDNAQRVAASGCGVRLDAPVRGEPLARALAQVLGDAAMAARCAQVRARMAAEPNGCDAAARFIERFAPGVAARRAQPA

SEQ ID NO: 9 - protein, 426 aa, Pseudomonas sp.
  SITE (1)..(426): Enyzme E2c
MHAILIAIGSAGDVFPFIGLARTLKLRGHRVSLCTIPVFRAAVEQHGIEFVPLSDELTYRRTMGDPRLWDPKTSFGVLWQAIAGMIEPVYEYVCAQRHDDIVVVGSLWALGARIAHEKYGIPYLSVQVSPSTLLSAHLPPVHPRFNVPEQVPLAMRKLLWRCIERFKLDRTCAPEINAVRRKVGLVGPAKRIFTQWMHSPQGVLCLFPAWFAPPQQDWPQPLHMTGFPLFDGSVPGTRLDDELQRFLEQGSRPLVFTQGSTEHLQGDFYAMALRALERLGARGIFLTGAGQEPLRGLPSHVLQRSYVPLGALLPACAGLVHPAGIGAMSLALAAGVPQVLLPCAHDQFDNAERLVRLGCGIRLGLPLREQALRESLWRLLEDPALAAACRRFMELSQPHSIACGKAAQVVERCHREGDVRWLKAAS

SEQ ID NO: 10 - protein, 426 aa, Pseudomonas sp.
  SITE (1)..(426): Enzyme E2d
MHAILIAIGSAGDVFPFIGLARTLKLRGHRVSLCTIPVFRDAVEQHGIAFVPLSDELTYRRTMGDPRLWDPKTSFGVLWQAIAGMIEPVYEYVSAQRHDDIVVVGSLWALGARIAHEKYGIPYLSAQVSPSTLLSAHLPPVHPKFNVPEQMPLAMRKLLWRCIERFKLDRTCAPEINAVRRKVGLETPVKRIFTQWMHSPQGVVCLFPAWFAPPQQDWPQPLHMTGFPLFDGSIPGTPLDDELQRFLDQGSRPLVFTQGSTEHLQGDFYAMALRALERLGARGIFLTGAGQEPLRGLPNHVLQRAYAPLGALLPSCAGLVHPGGIGAMSLALAAGVPQVLLPCAHDQFDNAERLVRLGCGMRLGVPLREQELRGALWRLLEDPAMAAACRRFMELSQPHSIACGKAAQVVERCHREGDARWLKAAS

SEQ ID NO: 11 - protein, 426 aa, Pseudomonas sp.
  SITE (1)..(426): Enzyme E2e
MHAILIAIGSAGDVFPFIGLARTLKLRGHRVSLCTIPVFRDAVEQHGIAFVPLSDELTYRRTMGDPRLWDPKTSFGVLWQAIAGMIEPVYEYVSAQRHDDIVVVGSLWALGARIAHEKYGIPYLSAQVSPSTLLSAHLPPVHPKFNVPEQMPLAMRKLLWRCIERFKLDRTCAPEINAVRRKVGLETPVKRIFTQWMHSPQGVVCLFPAWFAPPQQDWPQPLHMTGFPLFDGSIPGTPLDDELQRFLDQGSRPLVFTQGSTEHLQGDFYAMALRALERLGARGIFLTGAGQEPLRGLPNHVLQRAYAPLGALLPSCAGLVHPGGIGAMSLALAAGVPQVLLPCAHDQFDNAERLVRLGCGMRLGVPLREQELRGALWRLLEDPAMAAACRRFMELSQPHSIACGKAAHVVERCHREGDARWLKAAS

SEQ ID NO: 12 - protein, 323 aa, Pseudomonas sp.
  SITE (1)..(323): Enzyme 3a
RIDMGVLVVLFNPGDDDLEHLGELAAAFPQLRFLAVDNSPHSDPQRNARLRGQGIAVLHHGNRQGIAGAFNQGLDALFRRGVQGVLLLDQDSRPGGAFLAAQWRNLQARNGQACLLGPRIFDRGDRRFLPAIHLDGLTLRQLSLDGLTTPQRTSFLISSGCLLTREAYQRLGHFDEELFIDHVDTEYSLRAQALDVPLYVDPRLVLEHRIGTRKTRRLGGLSLSAMNHAPLRRYYLARNGLLVLRRYARSSPLALLANLPTLTQGLAVLLLERDKLLKLRCLGWGLWDGLRGRGGALETNRPRLLKRLAGPAVASVASGKAKA

SEQ ID NO: 13 - protein, 321 aa, Burkholderia sp.
  SITE (1)..(321): Enzyme 3b
MTILGALVILYDPTDEQLSGLEALARDSDALVVVDNTPHEHAAARERVRALSARTNTVWRHHGNRGGVAGGYNAGLSVLFAQGVEAVALFDQDSTVPAGYFERMREACAQLGEQPGAHAGAFIAGPRIYDANEQRFLPELMTSGVTVRRVRVEGETAPQRCAFLISSGSVISRAAYARLGRFDEALFIDHVDTEYCLRALAHNVPLYVVPPLVLTHRIGARRRHKVGPFELTAMHHGWLRRYYGARNAMQLGLQYGLRFPVALVPNLLTIWQVIQVVLCEREKGAKLRGIALGVLDGLFGRLGSFDDARAGAAAREPVRQE

SEQ ID NO: 14 - protein, 325 aa, Pseudomonas sp.
  SITE (1)..(325): Enzyme E3c
MDRIDMGVLVVLFNPGDDDLEHLGELAAAFPQLRFLAVDNSPHSDPQRNARLRGQGIAVLYHGNRQGIAGAFNQGLDTLFRRGLQGVLLLDQDSRPGGAFLAAQWRNLQACNGQACLLGPRIFDRGDRRFLPAIHLDGLALRQLSLDGLTTPQRTSFLISSGCLLTREAYQRLGHFDEELFIDHVDTEYSLRAQALDVPLYVDPRLVLEHRIGTRKTRRLGGLSLSAMNHAPLRRYYLARNGLLVLRRYARSSPLALLANLPTLTQGLAVLLLERDKLLKLRCLGWGLWDGLRGRGGALERNRPRLLKRLAGPAVAPTVPGKAKA

SEQ ID NO: 15 - protein, 325 aa, Pseudomonas sp.
  SITE (1)..(325): Enzyme E3d
MDRIDMGVLVVLFNPGDDDLEHLGELAAAFPQLRFLAVDNSPHSDPQRNARLRGQGIAVLHHGNRQGIAGAFNQGLDALFRRGVQGVLLLDQDSRPGGAFLAAQWRNLQARNGQACLLGPRIFDRGDRRFLPAIHLDGLTLRQLSLDGLTTPQRTSFLISSGCLLTREAYQRLGHFDEELFIDHVDTEYSLRAQALDVPLYVDPRLVLEHRIGTRKTRRLGGLSLSAMNHAPLRRYYLARNGLLVLRRYARSSPLALLANLPTLTQGLAVLLLERDKLLKLRCLGWGLWDGLRGRGGALERNRPRLLKRLAGPAVASVASGKAKA

SEQ ID NO: 16 - protein, 422 aa, Pseudomonas sp.
  SITE (1)..(422): Enzyme E8a
VSTTSLCPSATREHGPGAKRVLPLLFLTCLLDAAGVGLIVPLLPTLIGSVAPLAVRDAATWGAALVMTFALLQLFFSPVLGSLSDRFGRRPVLVLAMLGFALSYLLLALADSLWMLFLGRALAGLTGASVATAMACAADLGTHGQRTRHFGWLYAGLALGMILGPALGGLLAVHGTTLPLLLAAGLCLLNALLAGLFLEETLPPTRRRRLDPRRMNALRSISGLARQPGVGRLLAVLALVFLGLQAVMVVWPFFVIEKFHWSSAWIGYSLALYGVLAVLAQTLGVNLCKRRLDDARLLRLGLALQGCGLLLFALVDSSFWLVCALLPFALGSLATPAMQGLLSARVPVDRQGELQGVLSSLMSLAAIVGPPLMSGLFHWGSGPLAPLPLAGAPFLAGALLVLAGLVLAWQLRPTGEERSWTG

SEQ ID NO: 17 - protein, 518 aa, Burkholderia sp.
  SITE (1)..(518): Enzyme E8b
MSADQAGVAPPAAAPLRGAKLALLTFALSLATFIEVLDSTVANVAVPAISGSLGVSNSQGTWVISSYSVAAAIAVPLTGWLARRVGELRLFVASVILFTLTSLLCGLARDLEVLVACRALQGLFSGPMVPLSQTILMRAFPPARRTLALALWGMTVLLAPIFGPVVGGWLIDNFSWPWIFLINLPIGLFSFAVCTLMLRPQAQRGEASPIDAPGIVLLVIGVGSLQAMLDLGHDRGWFDSPLITALAIAAGVSLVSLLIWELGEAHPVVDLSLFRERTFTFCVVIISLGMMSFSVVGVVFPLWLQAVMGYTAYQAGLATASMGVLALVFSILVGLYASRVDARVLVTFGFGVFAAVMWWSTHFTLSMTFAQVVTPRLIQGMGLPCFFIPLTAATLSRVPDEKLAAASSLSNFLRTLSAAFGTALSVTWWDNRATYHYAVVSQSVTRASENTQRYVDALHAMGLHGARELSSLHQVVRQQAYMMATNDMFYMASATCLLLAGLMWLTRPKRGAAAALGH

SEQ ID NO: 18 - protein, 553 aa, Burkholderia sp.
  SITE (1)..(553): Enzyme E8c
MRARARRRASRCGRNERNGPQRDTGKQEGRIIRMTQTATQAATRAMIATGSRAARRLAAAALAWALAGCVPSGFEPALAPRTPGDDALAHTAGGAAHGAWPSPDWVRQLGDPQLDALVDEALRQNPTLQAAQARIGVAQSQLQQFESLTGLTATAGASLSKAHVPRSGGTINTTFNGLPVSVPLVGESVVSSSSLFVGLNYQLDLWGKNAAATRGLLSMRDAARVEAEQARLALSVAIVTLYGELDRAYALRELLQQKRRASEQVETVLRERAARGIDNGYDADDAALKRGKLLEQLALTDEQIQLQKLQLGVLSGRGPERGLSLARPKLAPLADAPLPARLPAGLLGRRPDIVAARLRVEAAYAAIDGTRASFYPDVNLAALGGLFALTPASLFKHDALGGSIGPALSLPIFDRGRLKAKLGGDVANADVALALYNQTVDAALGEVARQLTSLSTVDALLEAQQQAVRSAQRMVALAQDRHRRGMGMRKDVNVAKLTLLDERAHVIELQARRRTLRVGLIGALGGGFDARPAGGAPLAQGKPFAAASDRPPD

SEQ ID NO: 19 - protein, 466 aa, Burkholderia sp.
  SITE (1)..(466): Enzyme E8d
MRPEATDTRRHRHQRHLHRVHERFNRHRPRASKPVGPIRDGLRAGPAVAGRRHRHHAREDLERYRHRYPAREGAHRSGRPRRRARAARAGARARIASAAGSRGDARRAPRDAPPALRAVLRAAGAGRADRGALLVRRRALQRGDGRRVRGRQRGADRRADPGDGDRRAGGGHAAGEGGAGAGEARRRGRVGGVRAGAGAARAGGAAGGEHAALDGDVRGDGEGARGGPEACAAGVSGGTGAAKVVAGERAGGAGGGAGAAGGGARAGQRAAGRAEPGGAAGGRAVQAGVPEPEAHDDRVAGGRHGRSAVGADRSAGGAGGAADVGGAVAAGVGGGELQGRADPAHAGGPAGAARIGPVRRAGDVPRPGGGGLGGHGQRVLDAAVAERGGELDQGGAAPAGGDLAGAVGAGGAPAAGGAVDARDGGDEGAWRPPARRRRAAAGAAHAGARSAGGRGRGRGFGSDSGE

SEQ ID NO: 20 - protein, 559 aa, Pseudomonas sp.
  SITE (1)..(559): Enzyme E9a
MSNKNNDELQRQASENTMGLNPVIGIRRKDLLSSARTVLRQAVRQPLHSAKHVAHFGLELKNVLLGKSSLAPDSDDRRFNDPAWSNNPLYRRYLQTYLAWRKELQDWVSSSDLSPQDISRGQFVINLMTEAMAPTNTLSNPAAVKRFFETGGKSLLDGLSNLAKDMVNNGGMPSQVNMDAFEVGKNLGTSEGAVVYRNDVLELIQYSPITEQVHARPLLVVPPQINKFYVFDLSPEKSLARFCLRSQQQTFIISWRNPTKAQREWGLSTYIDALKEAVDAVLSITGSKDLNMLGACSGGITCTALVGHYAALGENKVNALTVLVSVLDTTMDNQVALFVDEQTLEAAKRHSYQAGVLEGSEMAKVFAWMRPNDLIWNYWVNNYLLGNEPPVFDILFWNNDTTRLPAAFHGDLIEMFKSNPLTRPDALKVCGTAIDLKQVKCDIYSLAGTNDHITPWPSCYRSAHLFGGKIEFVLSNSGHIQSILNPPGNPKARFMTGADRPGDPVAWQENAIKHADSWWLHWQSWLGERAGALKKAPTRLGNRTYAAGEASPGTYVHER

SEQ ID NO: 21 - protein, 560 aa, Pseudomonas sp.
  SITE (1)..(560): Enzyme E9b
MTDKPAKGSTTLPATRMNVQNAILGLRGRDLLSTLRNVGRHGLRHPLHTAHHLLALGGQLGRVMLGDTPYQPNPRDARFSDPTWSQNPFYRRGLQAYLAWQKQTRQWIDESHLNDDDRARAHFLFNLINDALAPSNSLLNPQAVKGLFNTGGQSLVRGVAHLLDDLRHNDGLPRQVDERAFEVGVNLAATPGAVVFRNELLELIQYSPMSEKQHARPLLVVPPQINRFYIFDLSATNSFVQYMLKSGLQVFMVSWSNPDPRHREWGLSSYVQALEEALNACRSISGNRDPNLMGACAGGLTMAALQGHLQAKKQLRRVRSATYLVSLLDSKFESPASLFADEQTIEAAKRRSYQRGVLDGGEVARIFAWMRPNDLIWNYWVNNYLLGKTPPAFDILYWNADSTRLPAALHGDLLEFFKLNPLTYASGLEVCGTPIDLQQVNIDSFTVAGSNDHITPWDAVYRSALLLGGERRFVLANSGHIQSIINPPGNPKAYYLANPKLSSDPRAWFHDAKRSEGSWWPLWLEWITARSGLLKAPRTELGNATYPLLGPAPGTYVLTR

SEQ ID NO: 22 - protein, 295 aa, Pseudomonas sp.
  SITE (1)..(295): Enzyme 10a
MRPEIAVLDIQGQYRVYTEFYRADAAENTIILINGSLATTASFAQTVRNLHPQFNVVLFDQPYSGKSKPHNRQERLISKETEAHILLELIEHFQADHVMSFSWGGASTLLALAHQPRYVKKAVVSSFSPVINEPMRDYLDRGCQYLAACDRYQVGNLVNDTIGKHLPSLLKRFNYRHVSSLDSHEYAQMHFHINQVLEHDLERALQGARNINIPVLFINGERDEYTTVEDARQFSKHVGRSQFSVIRDAGHFLDMENKTACENTRSVMLGFLKPTVREPRQRYQPVQQGQHALAI

SEQ ID NO: 23 - protein, 300 aa, Pseudomonas sp.
  SITE (1)..(300): Enzyme 10b
MRPETAIIEIHGQYRIHTEFYGNPAAQQTIILVNGSLSTTASFAQTVKYLQPHYNVVLYDQPYAGQSKPHNENHTPISKECEARILLELIERFRAEVVMSFSWGGVATLLALAQRPGRIRRAVVNSFSPQLNPAMLDYLHRGLDYLAACDRTQIGNLVNETIGRYLPQLFKRYNFRHVSSLDEHEYHQMHFHIREVLRLNADSYTESFAGIEIPMLFMNGELDIYTTPHEARQFGQLIRGAEFHTIRNAGHFIDVEHKAAWQQTQDALLAFLRPQRTQPLNPIYRPQPNGASVPLAALAS

(19) United States
(12) Patent Application Publication
(10) Pub. No.: US 2018/0066297 A1
(43) Pub. Date: Mar. 8, 2018

(54) RHAMNOLIPID SYNTHESIS

(71) Applicants: Thomas Haas, Münster (DE); Thomas Bülter, Duisburg (DE); Stefan Buchholz, Hanau (DE); Simon Beck, Münster (DE)

(72) Inventors: Thomas HAAS, Münster (DE); Thomaas BÜLTER, Duisburg (DE); Stefan BUCHHOLZ, Hanau (DE); Simon BECK, Münster (DE)

(73) Assignee: Evonik Degussa GmbH, Essen (DE)

(21) Appl. No.: 15/551,904

(22) PCT Filed: Feb. 16, 2016
(86) PCT No.: PCT/EP2016/053222

§ 371 Date: Aug. 18, 2017

(30) Foreign Application Priority Data
Feb. 19, 2015 (EP) 15155706.3

(51) Int. Cl. C12P 19/44; C12N 9/10; C12N 9/14

(52) U.S. Cl. CPC C12P 19/44; C12N 9/14; C12N 9/1051

(57) ABSTRACT

There is provided a method of producing at least one rhamnolipid comprising:
(a) contacting a recombinant cell with a medium containing a carbon source;
wherein the recombinant cell has been genetically modified such that, compared to the wild-type of the cell, the cell has an increased activity of at least one of the enzymes E₁, E₂ and E₃, wherein the enzyme E₁ is an α/β hydrolase (RHIA), the enzyme E₂ is a rhamnosyltransferase I (RHIB) and the enzyme E₃ is a rhamnosyltransferase II (RHIC), and
wherein the carbon source is an alkane and/or alkanoic acid comprising 6 to 10 carbon atoms.

### FIELD OF THE INVENTION

The present invention relates to methods and cells for producing at least one rhamnolipid from a carbon source. In particular, the carbon source may an organic compound comprising at least 6 carbon atoms and the rhamnolipid may be a dirhamnosyl lipid.

### BACKGROUND OF THE INVENTION

There is a general demand in the market for biodegradable surfactants that are produced from renewable raw materials as a suitable alternative to the currently available surfactants which are obtained from petrochemical raw materials. This demand is in particular accentuated with the foreseeable shortage of petrochemical raw materials and increasing demand for surfactants. Rhamnolipids are at least one example of such a surfactant. Rhamnolipids represent an economically interesting class because they may potentially replace conventional surfactants made from petroleum or products thereof, and thus invariably improve the environmental performance of the resulting formulations.

These rhamnolipids comprise at least one monorhamnosyl lipid or two rhamnose radicals (dirhamnosyl lipids) and one or two 3-hydroxy fatty acid residues (Handbook of Hydrocarbon and Lipid Microbiology, 2010). They have surface-active properties, which are needed in all sorts of applications for use as a surfactant (see Leitermann et al., 2009). In particular, rhamnolipids may be employed to a large extent as surfactants in household, cleaning, cosmetic, food processing, pharmaceutical, plant protection and other applications.

The currently used methods to produce these rhamnolipids employ wild-type isolates of various human and animal pathogenic bacteria, particularly members of the genera Pseudomonas and Burkholderia, (Handbook of Hydrocarbon and Lipid Microbiology, 2010). The fact that these pathogenic organisms are capable of causing diseases to the consumer considerably reduces the customer's acceptance for these conventionally produced rhamnolipids. Further, higher safety requirements also increase the production costs owing to increased capital expenditure and possibly additional production steps. Since the products in which these rhamnolipids are used are mostly high volume chemicals which can be produced at very low costs, the rhamnolipids must also be able to be produced at costs as low as possible, without health risks for the customer and with defined properties as far as possible.

The current methods available for production of rhamnolipids include the use of these pathogenic organisms and vegetable oils as the sole or co-substrate (Handbook of Hydrocarbon and Lipid Microbiology, 2010). Vegetable oils, however, are comparatively expensive raw materials in comparison to other carbon sources, such as, for example, glucose, sucrose or polysaccharides such as, for example, starch, cellulose and hemicellulose, glycerol, CO, CO2 or CH₄. Rhamnolipids are also produced by non-pathogenic organisms using carbon sources, such as, for example, glucose, sucrose or polysaccharides as taught in WO2012013554A1. A lot of resources are needed to build rhamnolipids from such short carbon chains.

However, there still lies a need to produce rhamnolipids (in particular, monorhamnosyl lipid and/or dirhamnosyl lipids) efficiently (i.e., inexpensively and, from the health point of view, safely) and in more than adequate amounts using non-pathogenic organisms and an alternative renewable raw material.

### DESCRIPTION OF THE INVENTION

The present invention relates to a method that may be capable of solving the problems present in the state of the art. In particular, the present invention relates to a method of producing at least one rhamnolipid by contacting a recombinant cell in the presence of at least one carbon source wherein the carbon source is at least one alkane comprising more than 6 carbon atoms. The recombinant cell comprises increased activity of at least one of the enzymes α/β hydrolase, rhamnosyltransferase I or rhamnosyltransferase II compared to the wild-type of the cell. This method may especially be advantageous as it may allow for high selective production of monorhamnosyl lipids and/or dirhamnosyl lipids with a reduction in the amount of undesirable by-products and intermediates produced. For example, there may at least be fewer intermediates such as dimers of β-Hydroxy fatty acids (fatty acid dimers) formed according to any aspect of the present invention compared to the currently available methods.

Further advantages of the method according to any aspect of the present invention include but are not limited to the fact that organisms can be utilised that are non-pathogenic and simple to culture. A further advantage may include the fact that with the method according to any aspect of the present invention, it may not be necessary that oils and simple carbohydrate substrates (e.g., glucose, fructose or sucrose) are the only substrate or co-substrate. According to any aspect of the present invention, another advantage may be that rhamnolipids having defined and modulatable properties can be produced. Also, specifically, dirhamnosyl lipids can be produced. A further advantage may be that rhamnolipids can be produced with higher space-time and carbon yields than with cells without enhancement of these activities.

Further, the method according to any aspect of the present invention enables higher carbon alkanes (C6 and above) to be directly converted to rhamnolipids without being first broken down to smaller C chains and then rebuilt into rhamnolipids.

According to any aspect of the present invention, rhamnolipids and/or rhamnolipid mixtures thereof that can be produced using any aspect of the present invention may be likewise a subject of the present invention. The rhamnolipids and mixtures that can be produced according to any aspect of the present invention can advantageously be employed at least in cleaning or care agents, in cosmetic, dermatological or pharmaceutical formulations as well as in plant protection formulations, surfactant concentrates and the like.

The term “care agents” is understood here as meaning a formulation that fulfills the purpose of maintaining an article in its original form, reducing or avoiding the effects of external influences (e.g., time, light, temperature, pressure, pollution, chemical reaction with other reactive compounds coming into contact with the article and the like) and aging, pollution, material fatigue, and/or even for improving desired positive properties of the article. An example of desired positive properties of the article may include features such as an improved hair gloss or a greater elasticity of the article and the like.

“Plant protection formulations” are to be understood herein as meaning those formulations that by the nature of their preparation are used for plant protection. This is in particular the case if at least one compound from the group consisting of herbicides, fungicides, insecticides, acaricides, nematicides, protective substances against bird damage, plant nutrients and soil structure-improving agents is contained in the formulation.

The rhamnolipids produced according to any aspect of the present invention may be used as a component of care and cleaning agents that are used in housekeeping, industry, in particular on hard surfaces, leather and/or textiles.

According to one aspect of the present invention, there is provided at least one method of producing at least one rhamnolipid comprising:

(a) contacting a recombinant cell with a medium containing a carbon source; wherein the recombinant cell has been genetically modified such that, compared to the wild-type of the cell, the cell has an increased activity of at least one of the enzymes E₁, E₂ and E_{3,} wherein the enzyme E₁ is an α/β hydrolase (RHIA), the enzyme E₂ is a rhamnosyltransferase I (RHIB) and the enzyme E₃ is a rhamnosyltransferase II (RHIC), and wherein the carbon source is an alkane comprising at least 6 or more carbon atoms. In particular, the cell may comprise increased activity of all three enzymes, E₁, E₂ and E₃. More in particular, the cell according to any aspect of the present invention comprises increased activity relative to the wild type cell of the enzyme E₁ an α/β hydrolase (RHIA), the enzyme E₂ a rhamnosyltransferase I (RHIB) and the enzyme E₃ a rhamnosyltransferase II (RHIC) and the rhamnolipid produced is at least one dirhamnosyl lipid.

According to another aspect of the present invention, there is provided a cell which is able to form at least one rhamnolipid from a C₆-C₁₀ alkane and/or alkanoic acid, wherein the cell has been genetically modified such that, compared to the wild-type of the cell, the cell has an increased activity of the enzyme oxidoreductase and at least one of the enzymes E₁, E₂ and E_{3,} wherein the enzyme E₁ is α/β hydrolase, the enzyme E₂ is rhamnosyltransferase I and the enzyme E₃ is rhamnosyltransferase II. In particular, the cell according to any aspect of the present invention comprises increased activity relative to the wild type cell of the enzyme E₁ an α/β hydrolase (RHIA), the enzyme E₂ a rhamnosyltransferase I (RHIB) and the enzyme E₃ a rhamnosyltransferase II (RHIC) and the rhamnolipid produced is at least one dirhamnosyl lipid.

More in particular, the cells according to any aspect of the present invention may be able to form rhamnolipids and compared to their wild-type have increased activity of at least one gene product or homologs of the gene products rhIA, rhIB and rhIC. At least in one example, the genes rhIA, rhIB and rhIC from Pseudomonas aeruginosa may be introduced into GRAS organisms (generally regarded as save) (as described in WO2012013554A1) to produce rhamnolipids from carbon source is an alkane comprising at least 6 or more carbon atoms. In one specific example the cell according to any aspect of the present invention may be P. putida of the strain KT2440.

In particular, the carbon source used in the method according to any aspect of the present invention is an alkane comprising at least 6 or more carbon atoms. The alkane may have the chemical formula CₙHₙ₊₂where ‘n’ may be more than 6. More in particular, ‘n’ may 6, 7, 8, 9, or 10. Even more in particular, the alkane may be selected from the group consisting of hexane, heptane, octane, nonane and decane.

In another example, the carbon source may be an alkanoic acid. In particular, the alkanoic acid may have 6 or more than 6 carbon atoms. More in particular, the alkanoic acid used in the method according to any aspect of the present invention may be selected from the group consisting of hexanoic acid, haptanoic acid, octanoic acid, nonanoic acid, decanoic acid and the like.

In one example, the carbon source may comprise a combination of an alkane and/or alkanoic acid with 6-10 carbon atoms. For example, the carbon source may comprise hexanoic acid and hexane; hexanoic acid and decane; hexanoic acid and decanoic acid; decanoic acid and hexane; decanoic acid and decane and the like.

The medium used according to any aspect of the present invention comprises at least one carbon source. The carbon source in the medium may at least be an alkane and/or alkanoic acid comprising 6 or more carbon atoms. In particular, the carbon source in the medium may consist essentially of or comprise substantially a hexane and/or hexanoic acid. In particular, the total amount of C₆ molecules is at least or equal to 20%, 40%, 50%, 60% or 70% by weight of the total carbon content in the medium of. More in particular, the total amount C₆ molecule is at least or equal to 50%, 70% or 80% by weight of the carbon source in the medium. Even more in particular, the C₆ molecule may at least be or equal to 90% or about 100% by weight of the carbon source in the medium. In this example, C₆ molecule may refer to hexane and/or hexanoic acid.

In another example, the carbon source in the medium may consist essentially of or comprise substantially a decane and/or decanoic acid. In particular, the total amount of C₁₀ molecules is at least or equal to 20%, 40%, 50%, 60% or 70% by weight of the total carbon content in the medium of. More in particular, the total amount C₁₀ molecule is at least or equal to 50%, 70% or 80% by weight of the carbon source in the medium. Even more in particular, the C₁₀ molecule may at least be or equal to 90% or about 100% by weight of the carbon source in the medium. In this example, C₁₀ molecule may refer to decane and/or decanoic acid.

In one example, the medium may comprise a second carbon source. In particular, the carbon source may be carbohydrates such as, for example, glucose, sucrose, arabinose, xylose, lactose, fructose, maltose, molasses, starch, cellulose and hemicellulose, vegetable and animal oils and fats such as, for example, soybean oil, safflower oil, peanut oil, hempseed oil, jatropha oil, coconut fat, calabash oil, linseed oil, corn oil, poppyseed oil, evening primrose oil, olive oil, palm kernel oil, palm oil, rapeseed oil, sesame oil, sunflower oil, grapeseed oil, walnut oil, wheat germ oil and coconut oil, fatty acids, such as, for example, caprylic acid, capric acid, lauric acid, myristic acid, palmitic acid, palmitoleic acid, stearic acid, arachidonic acid, behenic acid, oleic acid, linoleic acid, linolenic acid, gamma-linolenic acid and its methyl or ethyl ester as well as fatty acid mixtures, mono-, di- and triglycerides containing any fatty acids mentioned above, alcohols such as, for example, glycerol, ethanol and methanol, hydrocarbons such as methane, carbon-containing gases and gas mixtures, such as CO, CO₂, synthesis or flue gas, amino acids such as L-glutamate or L-valine or organic acids such as, for example, acetic acid. These substances can be used individually or as a mixture. Carbohydrates, in particular monosaccharides, oligosaccharides or polysaccharides, as the carbon source as is described in U.S. Pat. No. 6,01,494 and U.S. Pat. No. 6,136,576 as well as of hydrocarbons, in particular of alkanes, alkenes and alkynes as well as the monocarboxylic acids derived therefrom and the mono-, di and triglycerides derived from these monocarboxylic acids, as well as of glycerol and acetate, may be used. Mono-, di- and triglycerides containing the esterification products of glycerol with caprylic acid, capric acid, lauric acid, myristic acid, palmitic acid, palmitoleic acid, stearic acid, arachidonic acid, behenic acid, oleic acid, linoleic acid, linolenic acid and/or gamma-linolenic acid may be used.

It is a great advantage according to any aspect of the present invention that the cells may be able to form rhamnolipids from the long chain carbon sources such as hexane, hexanoic acid, decane, decanoic acid and the like. This is because these long carbon chains can directly be used in the skeleton of the rhamnolipids formed. This saves the resources and energy needed to break these long chain molecules to C2 molecule blocks that are then used to build complex rhamnolipids. The method according to any aspect of the present invention thus is more energy efficient and reduces waste. In particular, the rhamnolipid produced is at least one dirhamnosyl lipid.

Basic compounds such as sodium hydroxide, potassium hydroxide, ammonia or ammonia water or acidic compounds such as phosphoric acid or sulfuric acid may be suitably employed in the medium for pH control of the culture. Anti-foam agents such as, for example, fatty acid polyglycol esters can be employed for the control of foam development. Suitable selectively acting substances such as, for example, antibiotics can be added to the medium for maintaining the stability of plasmids. To maintain aerobic conditions, oxygen or oxygen-containing gas mixtures such as, for example, air may be incorporated into the culture.

The temperature of the culture is usually more than or equal to 20° C., 25° C., it can also be more than or equal to 40° C., wherein advantageously a culturing temperature of at least or equal to 95° C., particularly at least or equal to 90° C. and more particularly at least or equal to 80° C. may be used.

A skilled person would understand what constitutes suitable conditions for producing rhamnolipids from a carbon source. The cells according to any aspect of the present invention may be cultured and/or grown in the carbon source to produce rhamnolipids. In another example, the recombinant cells according to any aspect of the present invention may just be in contact with the carbon source without growing any further. The recombinant cells according to any aspect of the present invention may produce rhamnolipids from at least an alkane and/or alkanoic acid comprising 6 to 10 carbon atoms. Using basic methods known in the art, a skilled person would be capable of varying the conditions in the medium to suit the relevant cell used according to any aspect of the present invention.

In the method according to any aspect of the present invention, the rhamnolipids formed by the cells can optionally be isolated from the cells and/or the medium. All methods known in the art for isolation of low molecular weight substances from complex compositions may be applied. For example, methods such as filtration, extraction, adsorption (chromatography), crystallization and the like may be used in the product phase.

The isolated product in the product phase may also comprise other unwanted residues of biomass and various impurities, such as oils, fatty acids and other nutrient media constituents. The separation of these impurities and the like may take place in a solvent-free process. Thus, for example, the isolated product may first be diluted with water to facilitate the adjustment of the pH. The product and aqueous phases may then be homogenized by converting the rhamnolipids into a water-soluble form by lowering or raising the pH with acids or alkalis respectively. The solubility of the rhamnolipids in the aqueous phase may be assisted by incubation of the reaction mixture at higher temperatures, e.g., at 60 to 90° C., and/or with constant mixing. By subsequent raising or lowering of the pH by alkalis or acids the rhamnolipids can then again be converted into a water-insoluble form, such that they can easily be separated from the aqueous phase. The product phase can then be washed once or several times with water to remove the water-soluble impurities.

Oil residues can be separated off, for example by extraction by means of suitable solvents advantageously by means of organic solvents. An alkane such as, for example, n-hexane and the like may be used as a solvent.

The separation of the product from the aqueous phase can be effected alternatively to the solvent-free process described above using a suitable solvent, e.g., an ester such as, for example, ethyl acetate, butyl acetate and the like. These extraction steps may be carried out in any desired sequence. A skilled person would be able to easily vary the sequence of steps and/or the solvents used to be suitable for the cell and the rhamnolipid to be extracted.

In another example, solvents may be employed in the extraction of the rhamnolipids produced according to any aspect of the present invention. In particular, organic solvents may be used. More in particular, n-Pentanol may be used as a solvent. A distillation, for example, takes place for the removal of the solvent. Subsequently, the lyophilized product can be further purified, for example by means of chromatographic methods. By way of example, precipitation by means of suitable solvents, extraction by means of suitable solvents, complexation, for example by means of cyclodextrins or cyclodextrin derivatives, crystallization, purification or isolation by means of chromatographic methods or conversion of the rhamnolipids into easily separable derivatives may be employed.

The recombinant cell employed according to any aspect of the present invention, has been genetically modified such that, compared to the wild-type of the cell, the cell has an increased activity of at least one of the enzymes E₁, E₂ and E₃, wherein the enzyme E₁ is an α/β hydrolase, the enzyme E₂ is a rhamnosyltransferase I and the enzyme E₃ is a rhamnosyltransferase II. The recombinant cell used according to any aspect of the present invention may be made according to the method disclosed in WO2012013554A1. In particular, the cell according to any aspect of the present invention comprises increased activity relative to the wild type cell of the enzyme E₁ an α/β hydrolase (RHIA), the enzyme E₂ a rhamnosyltransferase I (RHIB) and the enzyme E₃ a rhamnosyltransferase II (RHIC).

In particular, in the cell according to any aspect of the present invention, the enzyme E₁ may be able to catalyze the conversion of 3-hydroxyalkanoyl-ACP via 3-hydroxyalkanoyl-3-hydroxyalkanoic acid-ACP to hydroxyalkanoyl-3-hydroxyalkanoic acid, the enzyme E₂ may be a rhamnosyltransferase I and may be able to catalyze the conversion of dTDP-rhamnose and 3-hydroxyalkanoyl-3-hydroxyalkanoate to a-L-rhamnopyranosyl-3-hydroxyalkanoyl-3-hydroxyalkanoate and the enzyme E₃ may be a rhamnosyltransferase II and may be able to catalyze the conversion of dTDP-rhamnose and a-L-rhamnopyranosyl-3-hydroxyalkanoyl-3-hydroxy-alkanoate to a-L-rhamnopyranosyl-(1-2)-a-L-rhamnopyranosyl-3-hydroxyalkanoyl-3-hydroxyalkanoate, wherein these enzymes E₁, E₂ and E₃ may be selected from the group consisting of:
at least one enzyme E₁ comprising an amino acid sequence selected from the group consisting of SEQ ID NO:2, SEQ ID NO:3, SEQ ID NO:4, SEQ ID NO:5, SEQ ID NO:6 and fragments thereof;
at least one enzyme E₂ comprising an amino acid sequence selected from the group consisting of SEQ ID NO:7, SEQ ID NO:8, SEQ ID NO:9, SEQ ID NO:10, SEQ ID NO:11 and fragments thereof, and
at least one enzyme E₃ comprising an amino acid sequence selected from the group consisting of SEQ ID NO:12, SEQ ID NO:13, SEQ ID NO:14, SEQ ID NO:15, and fragments thereof. The fragment with respect to any one of the enzymes E₁, E₂, or E₃ may comprise a polypeptide sequence in which up to 25% of the amino acid radicals are modified by deletion, insertion, substitution or a combination thereof compared to the sequence of the respective enzyme and the fragment comprises at least 10% of the enzymatic activity of the respective enzyme.

In particular, the enzyme E₁ in the cell according to any aspect of the present invention may be selected from the group consisting of:
an enzyme E₁ₐ comprising a polypeptide sequence SEQ ID NO:2 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:2 by deletion, insertion, substitution or a combination thereof and that still has at least 10%, 50%, 80%, in particular more than 90% of the enzymatic activity of the enzyme having the reference sequence SEQ ID NO:2, wherein enzymatic activity for an enzyme E₁ₐ may be understood as meaning the ability to convert 3-hydroxydecanoyl-ACP via 3-hydroxydecanoyl-3-hydroxydecanoic acid-ACP to hydroxydecanoyl-3-hydroxydecanoic acid,
an enzyme E_{1b} comprising a polypeptide sequence SEQ ID NO:3 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:3 by deletion, insertion, substitution or a combination thereof and that still has at least 10%, 50%, 80%, in particular more than 90% of the enzymatic activity of the enzyme having the reference sequence SEQ ID NO:3, wherein enzymatic activity for an enzyme E_{1b} may be understood as meaning the ability to convert 3-hydroxydecanoyl-ACP via 3-hydroxydecanoyl-3-hydroxydecanoic acid-ACP to hydroxydecanoyl-3-hydroxydecanoic acid,
an enzyme E_{1c} comprising a polypeptide sequence SEQ ID NO:4 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:4 by deletion, insertion, substitution or a combination thereof and that still has at least 10%, 50%, 80%, in particular more than 90% of the enzymatic activity of the enzyme having the reference sequence SEQ ID NO:4, wherein enzymatic activity for an enzyme E_{1c} may be understood as meaning the ability to convert 3-hydroxydecanoyl-ACP via 3-hydroxydecanoyl-3-hydroxydecanoic acid-ACP to hydroxydecanoyl-3-hydroxydecanoic acid,
an enzyme E_{1d} comprising a polypeptide sequence SEQ ID NO:5 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:5 by deletion, insertion, substitution or a combination thereof and that still has at least 10%, 50%, 80%, in particular more than 90% of the enzymatic activity of the enzyme having the reference sequence SEQ ID NO:5, wherein enzymatic activity for an enzyme E_{1d} may be understood as meaning the ability to convert 3-hydroxydecanoyl-ACP via 3-hydroxydecanoyl-3-hydroxydecanoic acid-ACP to hydroxydecanoyl-3-hydroxydecanoic acid, and
an enzyme E₁ₑ comprising a polypeptide sequence SEQ ID NO:6 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:6 by deletion, insertion, substitution or a combination thereof and that still has at least 10%, 50%, 80%, in particular more than 90% of the enzymatic activity of the enzyme having the reference sequence SEQ ID NO:6, wherein enzymatic activity for an enzyme E₁ₑ may be understood as meaning the ability to convert 3-hydroxydecanoyl-ACP via 3-hydroxydecanoyl-3-hydroxydecanoic acid-ACP to hydroxydecanoyl-3-hydroxydecanoic acid.

In particular, the enzyme E₂ used in the cell according to any aspect of the present invention may be selected from the group consisting of:
an enzyme E₂ₐ having polypeptide sequence SEQ ID NO:7 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:7 by deletion, insertion, substitution or a combination thereof and that still has at least 10%, 50%, 80%, in particular more than 90% of the enzymatic activity of the enzyme having the reference sequence SEQ ID NO:7, wherein enzymatic activity for an enzyme E₂ₐ may be understood as meaning the ability preferably to convert dTDP-rhamnose and 3-hydroxydecanoyl-3-hydroxydecanoic acid to a-L-rhamnopyranosyl-3-hydroxydecanoyl-3-hydroxydecanoic acid,
an enzyme E_{2b} having polypeptide sequence SEQ ID NO:8 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:8 by deletion, insertion, substitution or a combination thereof and that still has at least 10%, 50%, 80%, in particular more than 90% of the enzymatic activity of the enzyme having the reference sequence SEQ ID NO:8, wherein enzymatic activity for an enzyme E_{2b} may be understood as meaning the ability preferably to convert dTDP-rhamnose and 3-hydroxydecanoyl-3-hydroxydecanoic acid to a-L-rhamnopyranosyl-3-hydroxydecanoyl-3-hydroxydecanoic acid,
an enzyme E_{2c} having polypeptide sequence SEQ ID NO:9 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:9 by deletion, insertion, substitution or a combination thereof and that still has at least 10%, 50%, 80%, in particular more than 90% of the enzymatic activity of the enzyme having the reference sequence SEQ ID NO:9, wherein enzymatic activity for an enzyme E_{2c} may be understood as meaning the ability preferably to convert dTDP-rhamnose and 3-hydroxydecanoyl-3-hydroxydecanoic acid to a-L-rhamnopyranosyl-3-hydroxydecanoyl-3-hydroxydecanoic acid,
an enzyme E_{2d} having polypeptide sequence SEQ ID NO:10 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:10 by deletion, insertion, substitution or a combination thereof and that still has at least 10%, 50%, 80%, in particular more than 90% of the enzymatic activity of the enzyme having the reference sequence SEQ ID NO:10, wherein enzymatic activity for an enzyme E_{2d} may be understood as meaning the ability preferably to convert dTDP-rhamnose and 3-hydroxydecanoyl-3-hydroxydecanoic acid to a-L-rhamnopyranosyl-3-hydroxydecanoyl-3-hydroxydecanoic acid, and
an enzyme E₂ₑ having polypeptide sequence SEQ ID NO:11 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:11 by deletion, insertion, substitution or a combination thereof and that still has at least 10%, 50%, 80%, in particular more than 90% of the enzymatic activity of the enzyme having the reference sequence SEQ ID NO:11, wherein enzymatic activity for an enzyme E₂ₑ may be understood as meaning the ability preferably to convert dTDP-rhamnose and 3-hydroxydecanoyl-3-hydroxydecanoic acid to a-L-rhamnopyranosyl-3-hydroxydecanoyl-3-hydroxydecanoic acid.

In particular, the enzyme E₃ used in the cell according to any aspect of the present invention may be selected from the group consisting of:
an enzyme E₃ₐ having polypeptide sequence SEQ ID NO:12 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:12 by deletion, insertion, substitution or a combination thereof and that still has at least 10%, 50%, 80%, in particular more than 90% of the enzymatic activity of the enzyme having the reference sequence SEQ ID NO:12, wherein enzymatic activity for an enzyme E₃ₐ may be understood as meaning the ability preferably to convert dTDP-rhamnose and a-L-rhamnopyranosyl-3-hydroxydecanoyl-3-hydroxydecanoic acid to a-L-rhamnopyranosyl-(1-2)-a-L-rhamnopyranosyl-3-hydroxydecanoyl-3-hydroxydecanoic acid,
an enzyme E_{3b} having polypeptide sequence SEQ ID NO:13 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:13 by deletion, insertion, substitution or a combination thereof and that still has at least 10%, 50%, 80%, in particular more than 90% of the enzymatic activity of the enzyme having the reference sequence SEQ ID NO:13, wherein enzymatic activity for an enzyme E_{3b} may be understood as meaning the ability preferably to convert dTDP-rhamnose and a-L-rhamnopyranosyl-3-hydroxydecanoyl-3-hydroxydecanoic acid to a-L-rhamnopyranosyl-(1-2)-a-L-rhamnopyranosyl-3-hydroxydecanoyl-3-hydroxydecanoic acid,
an enzyme E_{3c} having polypeptide sequence SEQ ID NO:14 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:14 by deletion, insertion, substitution or a combination thereof and that still has at least 10%, 50%, 80%, in particular more than 90% of the enzymatic activity of the enzyme having the reference sequence SEQ ID NO:14, wherein enzymatic activity for an enzyme E_{3c} may be understood as meaning the ability preferably to convert dTDP-rhamnose and a-L-rhamnopyranosyl-3-hydroxydecanoyl-3-hydroxydecanoic acid to a-L-rhamnopyranosyl-(1-2)-a-L-rhamnopyranosyl-3-hydroxydecanoyl-3-hydroxydecanoic acid, and
an enzyme E_{3d} having polypeptide sequence SEQ ID NO:15 or having a polypeptide sequence in which up to 25%, 20%, 15% in particular up to 10, 9, 8, 7, 6, 5, 4, 3, 2, 1% of the amino acid radicals are modified compared to the reference sequence SEQ ID NO:15 by deletion, insertion, substitution or a combination thereof and that still has at least 10%, 50%, 80%, in particular more than 90% of the enzymatic activity of the enzyme having the reference sequence SEQ ID NO:15, wherein enzymatic activity for an enzyme E_{3d} may be understood as meaning the ability preferably to convert dTDP-rhamnose and a-L-rhamnopyranosyl-3-hydroxydecanoyl-3-hydroxydecanoic acid to a-L-rhamnopyranosyl-(1-2)-a-L-rhamnopyranosyl-3-hydroxydecanoyl-3-hydroxydecanoic acid.

A skilled person would understand that the activities indicated above for the enzymes E₁ₐ to E_{3b} are only special exemplary choices of a broader spectrum of activities of these enzymes; the respective activity mentioned is that for which a reliable measuring method is available in the case of a given enzyme. Thus, it is obvious that an enzyme with a substrate having an unbranched, saturated C₁₀-alkyl radical may also be able to convert those substrates that contain a C₆- or C₁₆ -alkyl radical, which can optionally also be branched or unsaturated.

The recombinant cell according to any aspect of the present invention may also be genetically modified such that compared to the wild-type of the cell, the cell has an increased activity of enzyme, oxidoreductase. In particular, the cell may be genetically modified such that the cell has increased activity of E₁, E₂ or E₃ or combinations thereof and oxidoreductase. More in particular, the cells may have increased activity of E₁, E₂, E₃ and oxidoreductase. In one example, the cells have increased activity of E₁ and E₂ and oxidoreductase, or E₁ and E₃ and oxidoreductase, or E₂ and E₃ and oxidoreductase.

The oxidoreductase may be an alkB-type oxidoreductase. This class of oxidoreductases, alkB, are redox proteins from the Pseudomonas putida AlkBGT system, dependent on two auxiliary polypeptides, alkG and alkT. AlkT is a FAD-dependent rubredoxin reductase transferring electrons from NADH to alkG. AlkG is a rubredoxin, an iron-containing redox protein functioning as a direct electron donor to alkB. In one particular example, the alkB-type oxidoreductase is alkB from Pseudomonas putida Gpo1 (accession number: CAB54050.1 (version 1), SEQ ID NO:1, any accession number used in the application refers to the respective sequence from the Genbank database run by the NCBl, wherein the release referred to is the one available online on the 4 Apr., 2014).

The enzyme alkB-type oxidoreductase has polypeptide sequence SEQ ID NO:1 or has a polypeptide sequence in which up to 25%, 20%, 15% in particular up to 10, 9, 8, 7, 6, 5, 4, 3, 2, 1% of the amino acid radicals are modified compared to the reference sequence SEQ ID NO:1 by deletion, insertion, substitution or a combination thereof and that still has at least 10%, 50%, 80%, in particular more than 92% of the enzymatic activity of the enzyme having the reference sequence SEQ ID NO:1, wherein enzymatic activity for an enzyme alkB-type oxidoreductase may be understood as meaning the ability to convert at least one alkane with 6 or more carbon atoms to the respective alkanoic acid according to any aspect of the present invention.

The oxidoreductase may be a monooxygenase. In particular, the monooxygenase may be a P450 type monooxygenase, e.g., cytochrome P450 from Candida tropicalis or from Cicer arietinum. More in particular, a CYP153 monooxygenase, e.g., cytochrome P450-monooxygenase from Alcanivorax borkumensis SK2 (YP_691921). The monooxygenase may be used in the first oxidation of butane to the alcohol.

In another example, the oxidoreductase may be an NAD(P)H dependent alcohol dehydrogenase (ADH). In particular, the ADH may be from Escherichia coli MS 187-1 (ZP_07145023), from Bacillus stearothermophilus (P42328), from Ralstonia eutropha (ACB78191.1), from Lactobacillus brevis (YP_795183.1), from Lactobacillus kefiri (ACF95832.1), from horse liver, from Paracoccus pantotrophus (ACB78182.1) or from Sphingobium yanoikuyae (EU427523.1). In one example, the ADH may be a flavin-dependent ADH, e.g., from Candida tropicalis (AAS46878.1).

In one example, the oxidoreductase may be from the glucose-methanol-choline-oxidoreductase family, especially from Caulobacter sp. K31 (ABZ74557.1). This particular oxidoreductase may also be used when a carbon source is an alkanoic acid comprising 6 to 10 carbon atoms according to any aspect of the present invention, directly or in situ produced from the respective alkane.

The term “increased activity of an enzyme” is understood as meaning increased intracellular activity.

The description and definitions below in relation to increasing the enzyme activity in cells apply both for the increase in the activity of the enzymes E₁ to E₃ and oxidoreductase as well as for all subsequently mentioned enzymes in this disclosure, the activity of which can optionally be increased. In particular, all the methods as described throughout this specification in relation to enzymes E₁, E₂ and E₃ may apply to the enzyme oxidoreductase that may be optionally present in the recombinant cell according to any aspect of the present invention.

In principle, an increase in the enzymatic activity can be achieved by increasing the copy number of the gene sequence or the gene sequences which code for the enzyme, using a strong promoter or an improved ribosome binding site, attenuating a negative regulation of gene expression, for example by transcription regulators, or amplifying a positive regulation of gene expression, modifying the codon usage of the gene, in various ways increasing the half-life of the mRNA or of the enzyme, modifying the regulation of the expression of the gene or utilizing a gene or allele that codes for an appropriate enzyme having an increased activity and optionally combining these measures. According to any aspect of the present invention, genetically modified cells are produced, for example, by transformation, transduction, conjugation or a combination of these methods using a vector that contains the desired gene, an allele of this gene or parts thereof and optionally contains a promoter making possible the expression of the gene. Heterologous expression is in particular achieved by integration of the gene or the alleles in the chromosome of the cell or an extrachromosomally replicating vector.

DE-A-10031999 gives several examples of ways to increase the enzyme activity in cells as exemplified by pyruvate carboxylase. A skilled person would easily be able to use the methods disclosed in DE-A-10031999 for increasing the enzyme activity in the cells according to any aspect of the present invention.

The expression of the above and all subsequently mentioned enzymes or genes is detectable with the aid of 1- and/or 2-dimensional protein gel separation and subsequent optical identification of the protein concentration in the gel using appropriate analytical software. If the increase in an enzyme activity is based exclusively on an increase in the expression of the corresponding gene, the quantification of the increase in the enzyme activity can be determined in a simple manner by a comparison of the 1- or 2-dimensional protein separations between wild-type and genetically modified cell. A customary method for the preparation of the protein gels in the case of corynebacterium and for the identification of the proteins is the procedure described by Hermann et al., 2001. The protein concentration may be analyzed by Western Blot hybridization using an antibody specific for the protein to be detected (Sambrook et al., 1989) and subsequent optical analysis using appropriate software for the concentration determination (Lohaus and Meyer, 1989). The activity of DNA-binding proteins can be measured by means of DNA band shift assays (also called gel retardation) (Wilson et al., 2001). The action of DNA-binding proteins on the expression of other genes can be detected by various well-known methods of the reporter gene assay (Sambrook et al., 1989). The intracellular enzymatic activities can also be determined according to various established methods (Donahue et al., 2000; Ray et al., 2000; Freed berg et al., 1973). If in the following examples no specific methods are indicated for the determination of the activity of a precise enzyme, the determination of the increase in the enzyme activity or the determination of the decrease of an enzyme activity may take place by means of methods described in Hermann et al., 2001, Lohaus et al., 1998, Lottspeich, 1999 and Wilson et al., 2001.

If the increase in the enzyme activity is accomplished by mutation of the endogenous gene, such mutations can be randomly produced either by conventional methods, such as, for example, by UV irradiation or by mutagenic chemicals, or selectively by means of genetic engineering methods such as deletion(s), insertion(s) and/or nucleotide exchange(s). Modified cells are obtained by these mutations. Mutants of enzymes are in particular also those enzymes that are no longer feedback-, product- or substrate-inhabitable or are so to a reduced degree at least in comparison to the wild-type enzyme.

If the increase in the enzyme activity is accomplished by increase in the synthesis of an enzyme, the copy number of the corresponding genes may be increased or the promoter and regulation region or the ribosome binding site, which is situated upstream of the structural gene, may be mutated. Expression cassettes, which are incorporated upstream of the structural gene, act in the same manner. It is also possible, by means of at least inducible promoters, to increase the expression the gene at any desired point in time. “Enhancers” may also be assigned to the enzyme gene of interest as regulatory sequences, which likewise bring about increased gene expression by means of an improved interaction between RNA polymerase and DNA. As a result of measures for the prolongation of the lifetime of the mRNA, the expression is likewise improved. Also, by prevention of the degradation of the enzyme protein the enzyme activity may also be increased. The genes or gene constructs are present here either in plasmids having a different copy number or are integrated and amplified in the chromosome. In another example, an overexpression of the genes concerned can be achieved by modification of the media composition and culture management. A person skilled in the art finds directions for this, inter alia, in Martin et al., 1987, Guerrero et al., 1994, Tsuchiya and Morinaga, 1988, Eikmanns et al., 1991, EP-A-0472869, U.S. Pat. No. 4,601,893, Schwarzer and Pühler, 1991, Reinscheid et al., 1994, LaBarre et al., 1993, WO96/15246A, Malumbres et al., 1993, JP10229891A, Jensen and Hammer, 1998 and in known textbooks of genetics and molecular biology. The measures described above likewise result in, like the mutations, to genetically modified cells that may be used in any aspect of the present invention.

Episomal plasmids, for example, are employed for increasing the expression of the respective genes. Suitable plasmids or vectors are in principle all theoretically available for this purpose to the person skilled in the art. Such plasmids and vectors can be taken, for example, from the brochures of companies Novagen, Promega, New England Biolabs, Clontech or Gibco BRL. In particular, plasmids and vectors can be found in: Glover, D. M., 1985, Rodriguez, R. L. and Denhardt, D. T., 1988, Butterworth, Stoneham; Goeddel, D. V., 1990, Fritsch, E. F. and Maniatis, T., 1989.

The plasmid vector, which comprises the gene to be amplified, is then converted to the desired strain by conjugation or transformation. The method of conjugation is described, for example, in Schäfer et al., 1994. Methods for transformation are described, for example at least in Thierbach et al., 1988, Dunican and Shivnan, 1989 and Tauch et al., 1994. After homologous recombination by means of a “cross-over” event, the resulting strain comprises at least two copies of the gene concerned. Using this method at least the copy number of the genes may be increased to a desired number in the strain.

Under the formulation used above and in the following examples “an activity of an enzyme (Eₓ) increased in comparison to its wild-type” is always to be understood as meaning an activity of the respective enzyme Eₓ increased by a factor of at least 2, particularly of at least 10, more particularly of at least 100, even more particularly of at least 1,000 and most particularly of at least 10,000. The cell according to any aspect of the present invention, which has “an increased activity of an enzyme (Eₓ) compared to its wild-type”, in particular also comprises a cell, whose wild-type contains no or at least no detectable activity of this enzyme Eₓ and which shows a detectable activity of this enzyme Eₓ only after increasing the enzyme activity, for example by overexpression. In this connection, the term “overexpression” or the formulation used in the following examples “increasing the expression” also comprises the case where a starting cell, for example a wild-type cell, has no or at least no detectable expression and a detectable synthesis of the enzyme Eₓ is induced only by recombinant methods. Eₓ may also refer to oxidoreductase.

“Wild-type” of a cell herein designates a cell, the genome of which is present in a state as is formed naturally by evolution. The term is used both for the entire cell as well as for individual genes. The term “wild-type” therefore in particular does not include those cells or those genes, the gene sequences of which have been modified at least partially by man by means of recombinant methods. The term ‘wild type’ may also include cells which have been genetically modified in other aspects (i.e., with regard to one or more genes) but not in relation to the genes of interest. The term “wild type” therefore does not include such cells where the gene sequences of the specific genes of interest have been altered at least partially by man using recombinant methods. Therefore, in one example, a wild type cell with respect to enzyme E₁ may refer to a cell that has the natural/non-altered expression of the enzyme E₁ in the cell. The wild type cell with respect to enzyme E₂, E₃, etc., may be interpreted the same way and may refer to a cell that has the natural/non-altered expression of the enzyme E₂, E₃, etc., respectively in the cell.

Changes of amino acid radicals of a given polypeptide sequence, which lead to no significant changes in the properties and function of the given polypeptide, are known to the person skilled in the art. Thus, for example, “conserved amino acids” can be mutually exchanged. Examples of such suitable amino acid substitutions include but are not limited to: Ala for Ser; Arg for Lys; Asn for GIn or His; Asp for Glu; Cys for Ser; GIn for Asn; Glu for Asp; Gly for Pro; His for Asn or GIn; Ile for Leu or Val; Leu for Met or Val; Lys for Arg or Gln or Glu; Met for Leu or Ile; Phe for Met or Leu or Tyr; Ser for Thr; Thr for Ser; Trp for Tyr; Tyr for Trp or Phe; Val for Ile or Leu. It is likewise known that changes, particularly at the N- or C-terminus of a polypeptide, in the form of, for example, amino acid insertions or deletions often exert no significant influence on the function of the polypeptide.

The activity of an enzyme can be determined by disrupting cells which contain this activity in a manner known to the person skilled in the art, for example with the aid of a ball mill, a French press or an ultrasonic disintegrator. Subsequently, the separation of cells, cell debris and disruption aids, such as, for example, glass beads, may be carried out by at least centrifugation for 10 minutes at 13,000 rpm and 4° C.

Using the resulting cell-free crude extract, enzyme assays with subsequent LC-ESI-MS detection of the products can then be carried out. Alternatively, the desired enzyme can be enriched by a means known to the person skilled in the art for example by chromatographic methods (such as nickel-nitrilotriacetic acid affinity chromatography, streptavidin affinity chromatography, gel filtration chromatography or ion-exchange chromatography) or else purified to homogeneity.

The activity of the enzyme E₁ may be determined using the enzyme samples obtained as described above in the following way: A standard assay may contain 100 μM E. coli ACP, 1 mM β-mercaptoethanol, 200 μM malonyl-coenzyme A, 40 μM octanoyl-coenzyme A (for E₁ₐ) or dodecanoyl-coenzyme A (for E_{1b}), 100 μM NADPH, 2 μg of E. coli FabD, 2 μg of Mycobacterium tuberculosis FabH, 1 μg of E. coli FabG, 0.1 M sodium phosphate buffer, pH 7.0, and 5 μg of enzyme E₁ in a final volume of 120 μL. ACP, β-mercaptoethanol and sodium phosphate buffer are incubated for 30 min at 37° C. to reduce the ACP completely. The reaction may then be started by addition of enzyme E₁. The reactions may be stopped using 2 ml of water, which has been acidified with HCl to pH 2.0, and subsequently extracted twice with 2 ml of chloroform/methanol (2:1 (v:v)). Phase separation is then carried out by centrifugation (16,100 g, 5 min, RT). The lower organic phase may be removed, evaporated completely in the vacuum centrifuge and the sediment may be taken up in 50 μl of methanol. Undissolved constituents are removed as sediments by centrifugation (16,100 g, 5 min, RT) and the sample is analyzed by means of LC-ESI-MS. The identification of the products takes place by analysis of the corresponding mass traces and the MS² spectra.

The activity of the enzyme E₂ may be determined as follows using the enzyme samples obtained as described above in the following way: A standard assay may contain 185 μl of 10 mM tris-HCl (pH 7.5), 10 μl of 125 mM dTDP-rhamnose and 50 μl of protein crude extract (about 1 mg of total protein) or purified protein in solution (5 μg of purified protein). The reaction is started by the addition of 10 μl of 10 mM ethanolic solution of 3-hydroxydecanoyl-3-hydroxydecanoic acid (for E₂ₐ) or 3-hydroxy-tetradecanoyl-3-hydroxytetradecanoic acid (for E_{2b}) and incubated for 1 h at 30° C. with shaking (600 rpm). Subsequently, the reaction may be treated with 1 ml of acetone. Undissolved constituents are removed as sediments by centrifugation (16,100 g, 5 min, RT) and the sample is analyzed by means of LC-ESI-MS. The identification of the products takes place by analysis of the corresponding mass traces and the MS² spectra.

The activity of the enzyme E₃ may be determined as follows using the enzyme samples obtained as described above: A standard assay may contain 185 μl of 10 mM tris-HCl (pH 7.5), 10 μl of 125 mM of dTDP-rhamnose and 50 μl of protein crude extract (about 1 mg of total protein) or purified protein in solution (5 μg of purified protein). The reaction is started by the addition of 10 μl of 10 mM ethanolic solution of a-L-rhamnopyranosyl-3-hydroxydecanoyl-3-hydroxydecanoic acid (for E₃ₐ) or a-L-rhamnopyranosyl-3-hydroxytetradecanoyl-3-hydroxytetradecanoic acid (for E_{3b}) and incubated for 1 h at 30° C. with shaking (600 rpm). Subsequently, the reaction is treated with 1 ml of acetone. Undissolved constituents are sedimented by centrifugation (16,100 g, 5 min, RT) and the sample is analyzed by means of LC-ESI-MS. The identification of the products takes place by analysis of the corresponding mass traces and the MS² spectra.

The recombinant cells according to any aspect of the present invention may have increased activities of at least E₁, E₂ and/or E₃. In particular, the cells may have increased activity of E₁, E₂ or E₃ or combinations thereof. More in particular, the cells may have increased activity of E₁, E₂ and E₃. In one example, the cells have increased activity of E₁ and E₂, or E₁ and E₃, or E₂ and E₃.

The activity of the enzyme oxidoreductase may be determined by any method known in the art. In particular, the activity of alkB-type oxidoreductase may be determined using the method disclosed in WO2009/077461A1, the activity of P450 type monooxygenases may be determined using the method provided in Scheps, D et al., 2011 and the activity of ADH by the method provided in Benson, S., Shapiro, J., J. Bacteriol. 1976, 126, 794-798.

The genetically modified cells according to any aspect of the present invention can be brought into contact with the medium continuously or discontinuously in the batch process (batch culture) or in the fed-batch process (feed process) or repeated fed-batch process (repetitive feed process) for the purpose of the production of the abovementioned products and thus cultured. A semi-continuous process is also conceivable, as is described in GB-A-1009370. A summary of known culturing methods is described in the textbook of Chmiel or in the textbook of Storhas. The culture medium to be used must satisfy in a suitable manner the demands of the respective strains. Descriptions of culture media of different yeast strains are contained, for example, in Klaus Wolf, 1996.

The cells according to any aspect of the present invention can be prokaryotes or eukaryotes. These can be mammalian cells (such as, for example, cells from man), plant cells or microorganisms such as yeasts, fungi or bacteria, wherein microorganisms are particularly preferred and bacteria and yeasts are most preferred.

Suitable bacteria, yeasts or fungi are in particular those bacteria, yeasts or fungi that are deposited in the Deutsche Sammlung von Mikroorganismen and Zellkulturen (German Collection of Microorganisms and Cell Cultures) GmbH (DSMZ), Brunswick, Germany, as bacterial, yeast or fungal strains. Bacteria suitable according to the invention belong to the genera that are listed under:
http://www.dsmz.de/species/bacteria.htm, yeasts suitable according to the invention belong to those genera that are listed under:
http://www.dsmz.de/species/yeasts.htm and fungi suitable according to the invention are those that are listed under:
http://www.dsmz.de/species/fungi.htm.

In particular, the cells may be selected from the genera Aspergillus, Corynebacterium, Brevibacterium, Bacillus, Acinetobacter, Alcaligenes, Lactobacillus, Paracoccus, Lactococcus, Candida, Pichia, Hansenula, Kluyveromyces, Saccharomyces, Escherichia, Zymomonas, Yarrowia, Methylobacterium, Ralstonia, Pseudomonas, Rhodospirillum, Rhodobacter, Burkholderia, Clostridium and Cupriavidus. More in particular, the cells may be selected from the group consisting of Aspergillus nidulans, Aspergillus niger, Alcaligenes latus, Bacillus megaterium, Bacillus subtilis, Brevibacterium flavum, Brevibacterium lactofermentum, Burkholderia andropogonis, B. brasilensis, B. caledonica, B. caribensis, B. caryophylli, B. fungorum, B. gladioli, B. glathei, B. glumae, B. graminis, B. hospita, B. kururiensis, B. phenazinium, B. phymatum, B. phytofirmans, B. plantarii, B. sacchari, B. singaporensis, B. sordidicola, B. terricola, B. tropics, B. tuberum, B. ubonensis, B. unamae, B. xenovorans, B. anthina, B. pyrrocinia, B. thailandensis, Candida blankii, Candida rugosa, Corynebacterium glutamicum, Corynebacterium efficiens, Escherichia coli, Hansenula polymorpha, Kluveromyces lactis, Methylobacterium extorquens, Paracoccus versutus, Pseudomonas argentinensis, P. borbori, P. citronellolis, P. flavescens, P. mendocina, P. nitroreducens, P. oleovorans, P. pseudoalcaligenes, P. resinovorans, P. straminea, P. aurantiaca, P. aureofaciens, P. chlororaphis, P. fragi, P. lundensis, P. taetrolens, P. antarctica, P. azotoformans, ‘P. blatchfordae’, P. brassicacearum, P. brenneri, P. cedrina, P. corrugata, P. fluorescens, P. gessardii, P. libanensis, P. mandelii, P. marginalis, P. mediterranea, P. meridiana, P. migulae, P. mucidolens, P. orientalis, P. panacis, P. proteolytica, P. rhodesiae, P. synxantha, P. thivervalensis, P. tolaasii, P. veronii, P. denitrificans, P. pertucinogena, P. cremoricolorata, P. fulva, P. monteilii, P. mosselii, P. parafulva, P. putida, P. balearica, P. stutzeri, P. amygdali, P. avellanae, P. caricapapayae, P. cichorii, P. coronafaciens, P. ficuserectae, ‘P. helianthi’, P. meliae, P. savastanoi, P. syringae, P. tomato, P. viridiflava, P. abietaniphila, P. acidophila, P. agarici, P. alcaliphila, P. alkanolytica, P. amyloderamosa, P. asplenii, P. azotifigens, P. cannabina, P. coenobios, P. congelans, P. costantinii, P. cruciviae, P. delhiensis, P. excibis, P. extremorientalis, P. frederiksbergensis, P. fuscovaginae, P. gelidicola, P. grimontii, P. indica, P. jessenii, P. jinjuensis, P. kilonensis, P. knackmussii, P. koreensis, P. lini, P. lutea, P. moraviensis, P. otitidis, P. pachastrellae, P. palleroniana, P. papaveris, P. peli, P. perolens, P. poae, P. pohangensis, P. psychrophila, P. psychrotolerans, P. rathonis, P. reptilivora, P. resiniphila, P. rhizosphaerae, P. rubescens, P. salomonii, P. segitis, P. septica, P. simiae, P. suis, P. thermotolerans, P. aeruginosa, P. tremae, P. trivialis, P. turbinellae, P. tuticorinensis, P. umsongensis, P. vancouverensis, P. vranovensis, P. xanthomarina, Ralstonia eutropha, Rhodospirillum rubrum, Rhodobacter sphaeroides, Saccharomyces cerevisiae, Yarrowia lipolytica and Zymomonas mobile. Even more in particular, the cells may be selected from the group consisting of Pseudomonas putida, Escherichia coli and Burkholderia thailandensis.

According to any aspect of the present invention, the cells in their wild-type may be incapable of forming detectable amounts of rhamnolipids and/or have none or no detectable activity of the enzymes E₁, E₂, E₃ and/or oxidoreductase.

It is advantageous according to any aspect of the present invention that the cell be able in its wild type to from polyhydroxyalkanoates having chain lengths of the mono-alkanoate of C6 to C₁₆. Such cells are, for example, Burkholderia sp., Burkholderia thailandensis, Pseudomonas sp., Pseudomonas putida, Pseudomonas aeruginosa, Pseudomonas oleovorans, Pseudomonas stutzeri, Pseudomonas fluorescens, Pseudomonas citronellolis, Pseudomonas resinovorans, Comamonas testosteroni, Aeromonas hydrophila, Cupriavidus necator, Alcaligenes latus and Ralstonia eutropha. In this connection, cells according to any aspect of the present invention may be genetically modified such that, compared to their wild-type, they are able to form fewer polyhydroxyalkanoates. Such cells are described, for example, at least in De Eugenio et al., 2010, and Rehm et al., 2001. Such a recombinant cell, able to form fewer polyhydroxyalkanoates compared to its wild-type, is in particular characterized in that, compared to its wild-type, it has a decreased activity of at least one enzyme E₉ or E₁₀.

E₉ represents a polyhydroxyalkanoate synthase, EC:2.3.1., in particular having polypeptide sequence SEQ ID NO:20 (E₉ₐ) or SEQ ID NO:21 (E_{9b}) or having a polypeptide sequence in which up to 25%, 20%, 15% in particular up to 10, 9, 8, 7, 6, 5, 4, 3, 2, 1% of the amino acid radicals compared to the respective reference sequence SEQ ID NO:20 or SEQ ID NO:21 are modified by deletion, insertion, substitution or a combination thereof and that still has at least 10%, 50%, particularly 80%, in particular more than 90% of the enzymatic activity of the enzyme having the respective reference sequence SEQ ID NO:20 or SEQ ID NO:21, wherein enzymatic activity for an enzyme E₉ (E₉ₐ and E_{9b}) may be understood as meaning the ability to convert 3-hydroxyalkanoyl-coenzyme A to poly-3-hydroxyalkanoic acid, in particular 3-hydroxytetradecanoyl-coenzyme A to poly-3-hydroxytetradecanoic acid.

E₁₀ represents a 3-hydroxyalkanoyl-ACP:coenzyme A transferase, in particular having polypeptide sequence SEQ ID NO:22 (E₁₀ₐ) or SEQ ID NO:23 (E_{10b})or having a polypeptide sequence in which up to 25%, 20%, particularly 15% in particular up to 10, 9, 8, 7, 6, 5, 4, 3, 2, 1% of the amino acid radicals are modified compared to the respective reference sequence SEQ ID NO:22 or SEQ ID NO:23 by deletion, insertion, substitution or a combination thereof and that still has at least 10%, 50%, particularly 80%, in particular more than 90% of the enzymatic activity of the enzyme having the respective reference sequence SEQ ID NO:22 (E₁₀ₐ) or SEQ ID NO:23 (E_{10b}), wherein enzymatic activity for an enzyme E₁₀ (E₁₀ₐ and E_{10b}) may be understood as meaning the ability to convert 3-hydroxyalkanoyl-ACP to 3-hydroxy-alkananoyl-coenzyme A, in particular 3-hydroxyalkananoyl-ACP to 3-hydroxytetradecanoyl-coenzyme A.

The activity of the enzyme E₉ (E₉ₐ and E_{9b}) may be determined for example by using the samples obtained as described above for the enzymes E₁ to E₃, by first mixing 560 μl of 100 mM tris/HCl, pH 7.5, 20 μl of 35 mM DTNB in DMSO and 20 μl of 41 mM 3-hydroxydecanoyl-coenzyme A. Subsequently, 5 μg of purified enzyme E₉ in 100 μl of tris/HCl, pH 7.5 are added, and subsequently the increase in the extinction at 412 nm (caused by addition of 5,5′-dithiobis(2-nitrobenzoate) (DTNB) to free SH groups) over time (ΔE/min) is recorded continuously for 1 min in a spectrophotometer.

The activity of the enzyme E₁₀ (E₁₀ₐ and E_{10b}) may be determined for example by using the samples obtained as described above for the enzymes E₁ to E₃. The standard assay may contain 3 mm MgCl2, 40 μm hydroxydecanoyl-coenzyme A and 20 μm E. coli ACP in 50 mm tris-HCl, pH 7.5, in a total volume of 200 μl. The reaction is started by addition of 5 μg of purified enzyme E₁₀ in 50 μl of tris/HCl, pH 7.5 and incubated for 1 h at 30° C. The reaction is stopped by addition of 50% (w/v) trichloroacetic acid and 10 mg/ml of BSA (30 μl). The released coenzyme A may be determined spectrophotometrically by recording the increase in the extinction at 412 nm, caused by addition of 5,5′-dithiobis(2-nitrobenzoate) (DTNB) to free SH groups, over time.

The phrase “decreased activity of an enzyme Eₓ” used with reference to any aspect of the present invention may be understood as meaning an activity decreased by a factor of at least 0.5, particularly of at least 0.1, more particularly of at least 0.01, even more particularly of at least 0.001 and most particularly of at least 0.0001. The phrase “decreased activity” also comprises no detectable activity (“activity of zero”). The decrease in the activity of a certain enzyme can be effected, for example, by selective mutation or by other measures known to the person skilled in the art for decreasing the activity of a certain enzyme.

In particular, the person skilled in the art finds instructions for the modification and decrease of protein expression and concomitant lowering of enzyme activity especially for Pseudomonas and Burkholderia, by means of interrupting specific genes, for example at least in Dubeau et al. 2009., Singh & Röhm. 2008., Lee et al., 2009 and the like.

Cells according to any aspect of the present invention are characterized in that the decrease in the enzymatic activity is achieved by modification of a gene comprising one of the nucleic acid sequences, wherein the modification is selected from the group comprising, consisting of, insertion of foreign DNA in the gene, deletion of at least parts of the gene, point mutations in the gene sequence, RNA interference (siRNA), antisense RNA or modification (insertion, deletion or point mutations) of regulatory sequences, such as, for example, promoters and terminators or of ribosome binding sites, which flank the gene.

Foreign DNA is to be understood in this connection as meaning any DNA sequence which is “foreign” to the gene (and not to the organism), i.e., endogenous DNA sequences can also function in this connection as “foreign DNA”. In this connection, it is particularly preferred that the gene is interrupted by insertion of a selection marker gene, thus the foreign DNA is a selection marker gene, wherein preferably the insertion was effected by homologous recombination in the gene locus.

In particular, the cells that may be used according to any aspect of the present invention may be Pseudomonas putida cells, which have a decreased polyhydroxyalkanoate synthesis compared to their wild-type. Such cells are described, for example, at least as KTOY01 and KTOY02 in Ren et al.,1998, Huisman et al., 1991, De Eugenio et al., 2010 and Ouyang et al. 2007.

The rhamnolipids formed according to the method of the present invention may at least be of the general formula (I) or its salt,

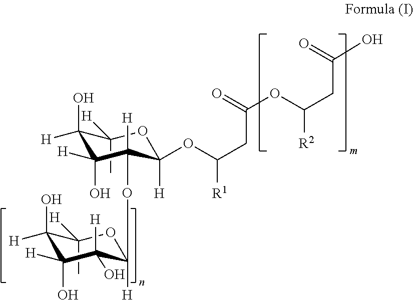

wherein

m=2, 1 or 0, in particular 1 or 0,

n=1 or 0, in particular 1,

R¹ and R²=independently of one another identical or different organic radical having 2 to 24, preferably 5 to 13 carbon atoms, in particular optionally branched, optionally substituted, in particular hydroxy-substituted, optionally unsaturated, in particular optionally mono-, di- or tri-unsaturated, alkyl radical, that may be selected from the group consisting of pentenyl, heptenyl, nonenyl, undecenyl and tridecenyl and (CH₂)ₒ-CH₃ with o=1 to 23, preferably 4 to 12.

In one example, the rhamnolipids formed according to the any aspect of the present invention may at least be of the general formula (I) or its salt, where n is 1. In particular, the rhamnolipid may be a dirhamnosyl lipid, also known as a dirhamnolipid. More in particular, the dirhamnosyl lipid may comprise the following formula (I):

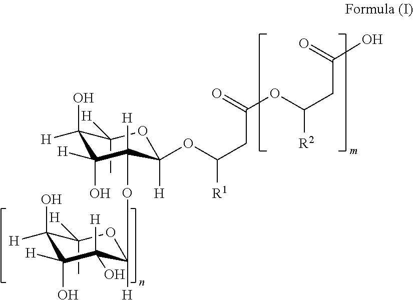

wherein

m=2, 1 or 0, in particular 1 or 0,

n=1

R¹ and R²=independently of one another identical or different organic radical having 2 to 24, preferably 5 to 13 carbon atoms, in particular optionally branched, optionally substituted, in particular hydroxy-substituted, optionally unsaturated, in particular optionally mono-, di- or tri-unsaturated, alkyl radical, that may be selected from the group consisting of pentenyl, heptenyl, nonenyl, undecenyl and tridecenyl and (CH₂)ₒ-CH₃ with o=1 to 23, preferably 4 to 12.

For the case where the cell according to any aspect of the invention is able to form a rhamnolipid having m=1, the radical may be

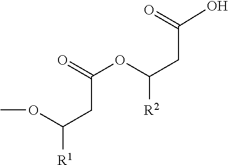

defined by means of R¹ and R² is derived from 3-hydroxyoctanoyl-3-hydroxyoctanoic acid, 3-hydroxyoctanoyl-3-hydroxydecanoic acid, 3-hydroxydecanoyl-3-hydroxyoctanoic acid, 3-hydroxyoctanoyl-3-hydroxydecenoic acid, 3-hydroxydecenoyl-3-hydroxyoctanoic acid, 3-hydroxyoctanoyl-3-hydroxydodecanoic acid, 3-hydroxydodecanoyl-3-hydroxyoctanoic acid, 3-hydroxyoctanoyl-3-hydroxydodecenoic acid, 3-hydroxydodecenoyl-3-hydroxyoctanoic acid, 3-hydroxydecanoyl-3-hydroxydecanoic acid, 3-hydroxydecanoyl-3-hydroxydecenoic acid, 3-hydroxydecenoyl-3-hydroxydecanoic acid, 3-hydroxydecenoyl-3-hydroxydecenoic acid, 3-hydroxydecanoyl-3-hydroxydodecanoic acid, 3-hydroxydodecanoyl-3-hydroxydecanoic acid, 3-hydroxydecanoyl-3-hydroxydodecenoic acid, 3-hydroxydecanoyl-3-hydroxytetradecenoic acid, 3-hydroxytetradecanoyl-3-hydroxydecenoic acid, 3-hydroxydodecenoyl-3-hydroxydecanoic acid, 3-hydroxydecanoyl-3-hydroxytetradecanoic acid, 3-hydroxytetradecanoyl-3-hydroxydecanoic acid, 3-hydroxydecanoyl-3-hydroxytetradecenoic acid, 3-hydroxytetradecenoyl-3-hydroxydecanoic acid, 3-hydroxydodecanoyl-3-hydroxydodecanoic acid, 3-hydroxydodecenoyl-3-hydroxydodecanoic acid, 3-hydroxydodecanoyl-3-hydroxydodecenoic acid, 3-hydroxydodecanoyl-3-hydroxytetradecanoic acid, 3-hydroxytetradecanoyl-3-hydroxydodecanoic acid, 3-hydroxytetradecanoyl-3-hydroxytetradecanoic acid, 3-hydroxyhexadecanoyl-3-hydroxytetradecanoic acid, 3-hydroxytetradecanoyl-3-hydroxyhexadecanoic acid or 3-hydroxyhexadecanoyl-3-hydroxyhexadecanoic acid.

It is obvious to the person skilled in the art that according to any aspect of the present invention, mixtures of different rhamnolipids of the general formula (I) may be formed. In one example, monolipids and dirhamnolipids may be formed. In particular, the rhamnolipid may be a mixture of at least one of the following rhamnolipids: 2RL-C8-C10, 1RL-C8-C10, 2RL-C10-C10, 1RL-C10-C10, 2RL-C10-C12:1, 1 RL-C10-C12:1, and 1 RL-C10-C12. In particular, the rhamnolipids according to any aspect of the present invention may be a dirhamnolipid/dirhamnosyl lipid selected from the group consisting of 2RL-C8-C10, 2RL-C10-C12:1, 2RL-C10-C10, combinations thereof and the like.

In this connection, the cells according to any aspect of the present invention may be able to form mixtures of rhamnolipids of the general formula (I), which are characterized in that in more than 80% by weight, more than 90% by weight, particularly more than 95% by weight of the rhamnolipids formed n is =1 and the radical defined by means of R¹ and R² is derived in less than 10% by weight, less than 5% by weight, particularly less than 2% by weight of the rhamnolipids formed, from 3-hydroxydecanoyl-3-hydroxyoctanoic acid or 3-hydroxyoctanoyl-3-hydroxydecanoic acid, wherein the % by weight indicated refers to the sum of all rhamnolipids of the general formula (I) formed.

Since the cells according to any aspect of the present invention can be used advantageously for the production of rhamnolipids and since these lipids are subsequently optionally purified, it is advantageous if the cells according to any aspect of the present invention have an increased activity compared to their wild-type of at least an enzyme E₈, which catalyzes the export of a rhamnolipid of the general formula (I) from the cell into the surrounding medium.

In this connection proteins E₈ are selected from the group consisting of an enzyme E₈ having polypeptide sequence SEQ ID NO:16 (E₈ₐ), SEQ ID NO:17 (E_{8b}), SEQ ID NO:18 (E_{8c}) or SEQ ID NO:19 (E_{8d}) or having a polypeptide sequence in which up to 25%, up to 20%, particularly up to 15% in particular up to 10, 9, 8, 7, 6, 5, 4, 3, 2, 1% of the amino acid radicals are modified by deletion, insertion, substitution or a combination thereof compared to the respective reference sequence SEQ ID NO:16 (E₈ₐ), SEQ ID NO:17 (E_{8b}), SEQ ID NO:18 (E_{8c}) or SEQ ID NO:19 (E_{8d}) and that still has at least 50%, 65%, particularly 80%, in particular more than 90% of the enzymatic activity of the enzyme having the respective reference sequence SEQ ID NO:16 (E₈ₐ), SEQ ID NO:17 (E_{8b}), SEQ ID NO:18 (E_{8c}) or SEQ ID NO:19 (E_{8d}), wherein enzymatic activity for an enzyme E₈ (E₈ₐ, E_{8b} E_{8c} and E_{8d}), is understood as meaning the ability to export a rhamnolipid of the general formula (I) from the cell into the surrounding medium.

### BRIEF DESCRIPTION OF THE FIGURES

No figures.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Pseudomonas putida Forming Rhamnolipids from Acetate

For the biotransformation of acetate to rhamnolipids a plasmid harboring Pseudomonas putida KT2440 strain was used. The plasmid pBBR1MCS-2::ABC is described in example 2 of DE 10 2010 032 484 A1 and the transformation of Pseudomonas putida KT2440 with the vector is described in Iwasaki et al. Biosci. Biotech. Biochem. 1994. 58(5): 851-854. The recombinant Pseudomonas putida KT2440 pBBR1MCS-2::ABC was cultivated on LB agar plates with 50 mg/l kanamycin.

For the preculture 10 ml of LB medium with 50 mg/I kanamycin in a 100 ml shaking flask were inoculated with a single colony from a fresh incubated agar plate and cultivated at 30° C. and 120 rpm for 15 h to an OD₆₀₀ₙₘ>3.5. Then the cell suspension was centrifuged, washed with fresh M9_BS_Ac medium and centrifuged again.

For the main culture 100 ml of fresh M9_BS_Ac medium (pH 7.4; 6.81 g/L Na₂HPO₄, 2.4 g/L KH₂PO_{4, 0.4} g/L NaCl, 1.4 g/L NH₄Cl, 2 ml/L 1 M MgSO₄×7 H₂O, 1.63 g/L ¹³C₂-Na-acetate, 0.13 ml/L 25% HCl, 1.91 mg/L MnCl₂×7 H₂O, 1.87 mg/L ZnSO₄×7 H₂O, 0.84 mg/L Na-EDTA×2 H₂O, 0.3 mg/L H₃BO₃, 0.25 mg/L Na₂MoO₄×2 H₂O, 4.7 mg/L CaCl₂×2 H₂O, 17.8 mg/L FeSO₄×7 H₂O, 0.15 mg/L CuCl₂×2 H₂O) in a 500 ml shaking flask were inoculated with centrifuged and washed cells from the preculture to an OD600nm of 0.12. This culture was incubated at 32° C. and 140 rpm for 142 h. After 6 h of cultivation, 2 g/L rhamnose was added to the culture for induction. After 7.5 h, 22.5 h, 30.5 h, 47.25 and 53 h of cultivation, 1 g/I ¹³C₂-Na-acetate were added respectively. At the start and during the culturing period, samples were taken. These were tested for optical density, pH and the different analytes (tested by NMR).

The results showed that in the main culture the amount of acetate decreased continuously from 1.63 g/I in the beginning to 1.3 g/I after 71.75 h (including the acetate feeding of 5 g/L¹³C₂-Na-acetate). Also, the concentration of rhamnolipids (2RL-C10-C10) (a dirhamnosyl lipid) was increased from 0.0 mg/I to 332 mg/I after 71.75 h of cultivation. The formed rhamnolipids were ¹³C-labeled (>90% in the fatty acid part). The carbon yield for ¹³C-labeled 2RL-C10-C10 a dirhamnosyl lipid was about 12.96% related to the consumed acetate and for non-labeled 2RL-C10-C10 it was 1.44%.

### Example 2

Pseudomonas putida Forming Rhamnolipids from Acetate and Decanoic Acid

For the biotransformation of acetate and decanoic acid to rhamnolipids a plasmid harboring Pseudomonas putida KT2440 strain was used. The plasmid pBBR1 MCS-2::ABC is described in example 2 of DE 10 2010 032 484 A1 and the transformation of Pseudomonas putida KT2440 with the vector is described in Iwasaki et al. Biosci. Biotech. Biochem. 1994. 58(5): 851-854. The recombinant Pseudomonas putida KT2440 pBBR1MCS-2::ABC was cultivated on LB agar plates with 50 mg/I kanamycin.

For the preculture 10 ml of LB medium with 50 mg/I kanamycin in a 100 ml shaking flask were inoculated with a single colony from a fresh incubated agar plate and cultivated at 30° C. and 120 rpm for 15 h to an OD600nm>3.5. Then the cell suspension was centrifuged, washed with fresh M9_BS_Ac medium and centrifuged again.

For the main culture 100 ml of fresh M9_BS_Ac medium (pH 7.4; 6.81 g/L Na₂HPO₄, 2.4 g/L KH₂PO₄, 0.4 g/L NaCl, 1.4 g/L NH₄Cl, 2 ml/L 1 M MgSO₄×7 H₂O, 1.63 g/L ¹³C₂-Na-acetate, 0.13 ml/L 25% HCl, 1.91 mg/L MnCl₂×7 H₂O, 1.87 mg/L ZnSO₄×7 H₂O, 0.84 mg/L Na-EDTA×2 H₂O, 0.3 mg/L H₃BO₃, 0.25 mg/L Na₂MoO₄×2 H₂O, 4.7 mg/L CaCl₂×2 H₂O, 17.8 mg/L FeSO₄×7 H₂O, 0.15 mg/L CuCl₂×2 H₂O) in a 500 ml shaking flask were inoculated with centrifuged and washed cells from the preculture to an OD₆₀₀ₙₘ of 0.12. This culture was incubated at 32° C. and 140 rpm for 142 h. After 6 h of cultivation, 2 g/L rhamnose were added to the culture for induction. After 22.5 h of cultivation, 1 g/L decanoic acid was added to the culture. After 7.5 h, 22.5 h, 30.5 h, 47.25 h and 53 h of cultivation, 1 g/I ¹³C₂-Na-acetate were added respectively. At the start and during the culturing period, samples were taken. These were tested for optical density, pH and the different analytes (tested by NMR).

The results showed that in the main culture the amount of acetate decreased continuously from 1.63 g/I in the beginning to 0 g/I after 71.75 h (including the acetate feeding of 5 g/L¹³C₂-Na-acetate). The concentration of decanoic acid decreased from 1 g/I at 22.5 h to 0 g/L after 71.75 h. Also, the concentration of rhamnolipids (2RL-C10-C10), a dirhamnosyl lipid was increased from 0.0 mg/I to 779 mg/I after 71.75 h of cultivation. The newly formed rhamnolipids were ¹³C-labeled (34% in the fatty acid part). The carbon yield for ¹³C-labeled 2RL-C10-C10, the dirhamnosyl lipid was about 6.05% based on the consumed acetate and decanoic acid and for non-labeled 2RL-C10-C10 it was 11.75%. This showed that a larger percentage of the resulting rhamnolipids were formed from the unlabeled decanoic acid than the acetate.

### Example 3

Pseudomonas putida Forming Rhamnolipids from Acetate and Hexanoic Acid

For the biotransformation of acetate and hexanoic acid to rhamnolipids a plasmid harboring Pseudomonas putida KT2440 strain was used. The plasmid pBBR1MCS-2::ABC is described in example 2 of DE 10 2010 032 484 A1 and the transformation of Pseudomonas putida KT2440 with the vector is described in Iwasaki et al. Biosci. Biotech. Biochem. 1994. 58(5): 851-854. The recombinant Pseudomonas putida KT2440 pBBR1MCS-2::ABC was cultivated on LB agar plates with 50 mg/I kanamycin.

For the preculture 10 ml of LB medium with 50 mg/I kanamycin in a 100 ml shaking flask are inoculated with a single colony from a fresh incubated agar plate and cultivated at 30° C. and 120 rpm for 15 h to an OD₆₀₀ₙₘ>3.5. Then the cell suspension is centrifuged, washed with fresh M9_BS_Ac medium and centrifuged again.

For the main culture 100 ml of fresh M9_BS_Ac medium (pH 7.4; 6.81 g/L Na₂HPO₄, 2.4 g/L KH₂PO₄, 0.4 g/L NaCl, 1.4 g/L NH₄Cl, 2 ml/L 1 M MgSO₄×7 H₂O, 1.63 g/L ¹³C₂-Na-acetate, 0.13 ml/L 25% HCl, 1.91 mg/L MnCl₂×7 H₂O, 1.87 mg/L ZnSO₄×7 H₂O, 0.84 mg/L Na-EDTA×2 H₂O, 0.3 mg/L H₃BO₃, 0.25 mg/L Na₂MoO₄×2 H₂O, 4.7 mg/L CaCl₂×2 H₂O, 17.8 mg/L FeSO₄×7 H₂O, 0.15 mg/L CuCl₂×2 H₂O) in a 500 ml shaking flask are inoculated with centrifuged and washed cells from the preculture to an OD₆₀₀ₙₘ of 0.12. This culture is incubated at 32° C. and 140 rpm for 142 h. After 6 h of cultivation, 2 g/L rhamnose are added to the culture for induction. After 22.5 h of cultivation, 1 g/L hexanoic acid is added to the culture. After 7.5 h, 22.5 h, 30.5 h, 47.25 h and 53 h of cultivation, 1 g/l ¹³C₂-Na-acetate are added respectively. At the start and during the culturing period, samples are taken. These are tested for optical density, pH and the different analytes (tested by NMR).

In the main culture the amount of acetate decreased continuously to 0 g/I after 71.75 h (including the acetate feeding of 5 g/L¹³C₂-Na-acetate). The concentration of hexanoic acid also decreased to 0 g/L after 71.75 h. Also, the concentration of rhamnolipid (2RL-C10-C10) increased during the cultivation. The newly formed rhamnolipids were ¹³C-labeled (<80% in the fatty acid part). The carbon yield for ¹³C-labeled 2RL-C10-C10, a dirhamnosyl lipid related to consumed acetate and hexanoic acid was lower and for non-labeled 2RL-C10-C10 it was higher than in cultures without hexanoic acid feeding. Again this confirmed the finding that a larger percentage of the resulting rhamnolipids were formed from the unlabeled hexanoic acid than the acetate.

1-11. (canceled)

12. A method of producing at least one rhamnolipid comprising contacting a recombinant cell with a medium containing a carbon source, wherein:
a) the recombinant cell has been genetically modified such that, compared to the wild-type cell, the recombinant cell has an increased activity of enzymes E₁, E₂ and E₃, wherein:
i) enzyme E₁ i is an α/β hydrolase (RHIA);
ii) enzyme E₂ is a rhamnosyltransferase I (RHIB);
iii) enzyme E₃ is a rhamnosyltransferase II (RHIC);
b) the carbon source is an alkane and/or alkanoic acid comprising 6 to 10 carbon atoms; and
c) the rhamnolipid comprises the general formula (I), 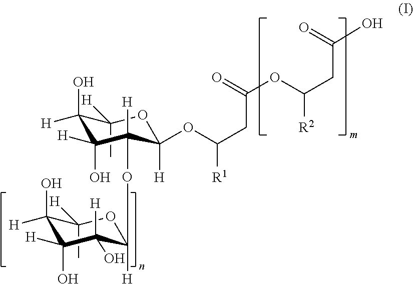
wherein
m=2, 1 or 0
n=1
R¹ and R²=independently of one another, identical or different organic radicals comprising 2 to 24 carbon atoms.

13. The method of claim 12, wherein said organic radicals are alkyl radicals that are optionally branched, optionally substituted, and optionally unsaturated.

14. The method of claim 13, wherein at least one alkyl radical is hydroxy-substituted.

15. The method of claim 13, wherein at least one alkyl radical is mono-, di- or tri-unsaturated.

16. The method of claim 12, wherein said identical or different organic radicals comprise 5 to 13 carbon atoms.

17. The method of claim 12, wherein said carbon source is an alkane selected from the group consisting of: hexane; heptane; octane; nonane; and decane; and/or an alkanoic acid selected from the group consisting of: hexanoic acid; haptanoic acid; octanoic acid; nonanoic acid; and decanoic acid.

18. The method of claim 12, wherein the recombinant cell has been genetically modified such that, compared to the wild-type cell, the recombinant cell has an increased activity of enzyme E₄, wherein E₄ is an oxidoreductase.

19. The method according to claim 18, wherein the oxidoreductase is selected from the group consisting of: alkB-type oxidoreductase; monooxygenase; and NAD(P)H dependent alcohol dehydrogenase (ADH).

20. The method of claim 19, wherein the carbon source is hexane and/or decane.

21. The method of claim 12, wherein at least 40% by weight of the total carbon content in the medium is hexane, decane, hexanoic acid and/or decanoic acid.

22. The method of claim 12, wherein:
a) enzyme E₁ is able to catalyse the conversion of 3-hydroxyalkanoyl-ACP via 3-hydroxyalkanoyl-3-hydroxyalkanoic acid-ACP to hydroxyalkanoyl-3-hydroxyalkanoic acid;
b) enzyme E₂ is able to catalyse the conversion of dTDP-rhamnose and 3-hydroxyalkanoyl-3-hydroxyalkanoate to α-L-rhamnopyranosyl-3-hydroxyalkanoyl-3-hydroxyalkanoate; and
c) enzyme E₃ is able to catalyse the conversion of dTDP-rhamnose and α-L-rhamnopyranosyl-3-hydroxyalkanoyl-3-hydroxyalkanoate to α-L-rhamnopyranosyl-(1-2)-α-L-rhamnopyranosyl-3-hydroxyalkanoyl-3-hydroxyalkanoate.

23. The method of claim 12, wherein:
a) enzyme E₁ comprises an amino acid sequence selected from the group consisting of: SEQ ID NO:2; SEQ ID NO:3; SEQ ID NO:4; SEQ ID NO:5; SEQ ID NO:6; and fragments thereof;
b) enzyme E₂ is selected from the group consisting of: SEQ ID NO:7; SEQ ID NO:8; SEQ ID NO:9; SEQ ID NO:10; SEQ ID NO:11; and fragments thereof; and
c) enzyme E₃ is selected from the group consisting of: SEQ ID NO:12; SEQ ID NO:13; SEQ ID NO:14; SEQ ID NO:15; and fragments thereof;
wherein said fragments comprise a polypeptide sequences in which up to 25% of the amino acid radicals are modified by deletion, insertion, substitution or a combination thereof compared to the sequence of the respective enzyme and the fragment comprises at least 10% of the enzymatic activity of the respective enzyme.

24. The method of claim 12, wherein the recombinant cell is selected from a genus of the group consisting of: Aspergillus; Corynebacterium; Brevibacterium; Bacillus, Acinetobacter; Alcaligenes; Lactobacillus; Paracoccus; Lactococcus; Candida; Pichia; Hansenula; Kluyveromyces; Saccharomyces; Escherichia; Zymomonas; Yarrowia; Methylobacterium; Ralstonia; Pseudomonas; Rhodospirillum; Rhodobacter; Burkholderia; Clostridium; and Cupriavidus.

25. The method of claim 12, wherein the recombinant cell is selected from the group consisting of: P. putida GPp121; P. putida GPp122; P. putida GPp123; P. putida GPp124; P. putida GPp104; P. putida KT42C1; P. putida KTOY01; and P. putida KTOY02.

26. The method of claim 12, wherein the rhamnolipid is a dirhamnosyl lipid selected from the group consisting of: 2RL-C10-C10; 2RL-C8-C10; 2RL-C10-C10; and 2RL-C10-C12:1.

27. The method of claim 12, wherein the recombinant cell has been genetically modified such that, compared to the wild-type cell, the recombinant cell has an increased activity of enzyme E₄, wherein E₄ is an oxidoreductase.

28. The method according to claim 27, wherein the oxidoreductase is selected from the group consisting of: alkB-type oxidoreductase; monooxygenase; and NAD(P)H dependent alcohol dehydrogenase (ADH).

29. The method of claim 28, wherein the recombinant cell is selected from the group consisting of: P. putida GPp121; P. putida GPp122; P. putida GPp123; P. putida GPp124; P. putida GPp104; P. putida KT42C1; P. putida KTOY01; and P. putida KTOY02.

30. The method of claim 29, wherein the rhamnolipid may be a dirhamnosyl lipid selected from the group consisting of 2RL-C10-C10, 2RL-C8-C10, 2RL-C10-C10 and 2RL-C10-C12:1.

31. The method of claim 30, wherein at least 40% by weight of the total carbon content in the medium is hexane, decane, hexanoic acid and/or decanoic acid.
